(19) United States
(12) Patent Application Publication
(10) Pub. No.: US 2021/0158378 A1
(43) Pub. Date: May 27, 2021

(54) METHOD AND SYSTEMS FOR PROVIDING AN UNEXPECTED REWARD FOR A MEASURED CHANGE OF A USER

(71) Applicant: TRUSX, INC., Waitsfield, VT (US)

(72) Inventor: Christopher Jones, Waitsfield, VT (US)

(21) Appl. No.: 17/119,538

(22) Filed: Dec. 11, 2020

Related U.S. Application Data
(63) Continuation-in-part of application No. 16/522,640, filed on Jul. 25, 2019.

(51) Int. Cl. G06Q 30/02; G06Q 10/06; G06N 3/04

(52) U.S. Cl. CPC G06Q 30/0201; G06N 3/04; G06Q 10/06393

(57) ABSTRACT

The present disclosure relates to systems and methods for providing a user with an unexpected reward communication for a measured change. The system generally includes at least one process configured for registering a user, determining a performance indicator for the first user, receiving performance data associated with the user, and sending a communication (e.g., an unexpected reward communication) to the user.

### CROSS-REFERENCE TO RELATED APPLICATIONS

This application is a Continuation in Part of U.S. Non-Provisional application Ser. No. 16/522,640 titled “Apparatus, code, methods, and systems for providing unexpected reward for a measured change of a user” and filed Jul. 25, 2019, the subject matter of which is incorporated herein by reference, which claims the benefit of U.S. Provisional Application Ser. No. 62/703,282 titled “APPARATUS, CODE, METHODS AND SYSTEMS FOR PROVIDING UNEXPECTED REWARDS FOR A MEASURED CHANGE OF A USER” and filed Jul. 25, 2018, now expired, the subject matter of which is incorporated herein by reference.

### STATEMENT REGARDING FEDERALLY SPONSORED RESEARCH OR DEVELOPMENT

Not applicable.

### INCORPORATION BY REFERENCE OF MATERIAL SUBMITTED ON A COMPACT DISC

Not applicable.

### TECHNICAL FIELD

The present invention relates to the field of electronically providing users with motivation and rewards to incentivize beneficial behavior.

### BACKGROUND

There is growing evidence that avoidance of weight gain may reduce the risk of certain cancers, especially post-menopausal breast cancer and aggressive variants of prostate cancer. In addition to benefits in cancer risk reduction, weight maintenance lowers the risk of other chronic diseases like heart disease and diabetes. Prior research has endeavored to identify approaches that encourage long-term increases in healthy behavior such as the consumption of fruits and vegetables. Baranowski et al. (2002) designed an intervention that awarded a special badge to participants who adopted healthier eating habits, showing that participants adopted a diet higher in fruit and low-fat vegetables immediately after the intervention; however, the diet was not maintained six months later. This study showed that behavior change is possible using even non-monetary incentives but underscores the need for alternative strategies to sustain long-term changes.

In addition to the U.S. population becoming progressively obese, the present healthcare system has swelled in terms of national spending to the point where it can no longer sustain itself. In response, the healthcare system is experiencing a monumental shift from volume to value. While it remains unclear as to how costs are going to be contained, one thing is certain: the diseases of today and the foreseeable future will continue to swell in the direction of chronic conditions that have unhealthy behavioral root causes. Health care delivery systems are going to need to respond to these behaviors in a more efficient manner, in order to ensure patients adopt a greater locus of accountability for the many complex aspects of health that can be modified.

To date, incentive-based research has largely taken the form of randomized controlled trials where patients are seen in weekly or monthly increments and rewarded using grant-funded cash incentives (e.g., $50 bills) contingent upon changes in behavior, like smoking cessation (Halpern et al. 2015) or kg of weight loss. One of the challenges is that this approach uses a “one size fits all” approach to incentivizing; when those studies are over, the money runs out and patients tend to relapse.

Behavioral interventions hold much promise for effectively changing physical activity behavior among cancer survivors. However, more research is needed to identify the best ways of supporting survivors to make and maintain these lifestyle changes. Tailored interventions for cancer survivors and at-risk individuals may be more effective when accounting for factors associated with health-promotion engagement. Identifying effective ways of assisting cancer survivors and at-risk individuals to adopt and maintain healthy lifestyles is important for enhancing the well-being and health outcomes of this group. There is a pressing need to direct more attention to this issue to inform the development of interventions that maintain healthy weight in order to avoid the downstream risk of chronic disease. While others have demonstrated that incentives work to motivate healthy behavioral change, traditional research programs that use incentives cost millions of dollars to implement and study.

As a result, there exists a need for improvements over the prior art and more particularly for a more efficient way of motivating people to change behavioral habits.

### SUMMARY

A system and method for providing a user an unexpected reward communication for a measured change is disclosed. This Summary is provided to introduce a selection of disclosed concepts in a simplified form that are further described below in the Detailed Description including the drawings provided. This Summary is not intended to identify key features or essential features of the claimed subject matter. Nor is this Summary intended to be used to limit the claimed subject matter's scope.

In one embodiment, a system for providing an unexpected reward communication for a measured change is disclosed. The system comprises one or more processors communicably connected to a communication network, where at least one processor is configured for: (a) receiving, over the communications network, from an information source computing device, training data comprising metrics associated with an attribute of the first user, (b) creating a first user record for the first user, (c) storing the training data in the first user record, (d) determining a performance indicator for the metrics associated with the attribute of the first user, (e) receiving, over the communications network, from the information source computing device, performance data comprising the metrics associated with the attribute of the first user, (f) sending, over the communications network, to a first user computing device a communication. If the performance data satisfies the performance indicator, the communication comprises an unexpected reward communication. Conversely, if the performance data does not satisfy the performance indicator, the communication comprises a loss aversion communication. Lastly, the receiving step (e) and sending step (0 may be repeated until the validated performance data satisfies the performance indicator.

In some embodiments, the system comprises at least one processor for receiving, over the communications network, from a validation computing device, validation data for validating at least a portion of the performance data associated with the attribute, determining if at least a portion of the performance data was validated using the validation data, and determining if the validated performance data satisfies the performance indicator. If the portion of the performance data was validated using the validation data, the portion of the performance data is the aforementioned validated performance data. In the event the validation steps are performed, they may also be repeated with the receiving step (e) and sending step (f) described above.

Additional aspects of the disclosed embodiment will be set forth in part in the description which follows, and in part will be obvious from the description, or may be learned by practice of the disclosed embodiments. The aspects of the disclosed embodiments will be realized and attained by means of the elements and combinations particularly pointed out in the appended claims. It is to be understood that both the foregoing general description and the following detailed description are exemplary and explanatory only and are not restrictive of the disclosed embodiments, as claimed.

### BRIEF DESCRIPTION OF THE DRAWINGS

The accompanying drawings, which are incorporated in and constitute part of this specification, illustrate embodiments of the invention and together with the description, serve to explain the principles of the disclosed embodiments. The embodiments illustrated herein are presently preferred, it being understood, however, that the invention is not limited to the precise arrangements and instrumentalities shown, wherein:

FIG. 1 is diagram of an operating environment that supports methods and systems for providing a user an unexpected reward for a measured change, according to an example embodiment;

FIG. 2 is a diagram of the operating environment in FIG. 1, illustrating the flow of data between the participating entities and components of the system, according to an example embodiment;

FIG. 3A is a block flow diagram according to an example embodiment for registering a user (e.g., first user, second user, third user, etc.), according to an example embodiment;

FIG. 3B is a block flow diagram for registering a first user, according to an example embodiment;

FIG. 3C is a block flow diagram for a method for providing a user an unexpected reward for a measuring change, according to an example embodiment;

FIG. 3D is a block flow diagram of a more particular method for providing a user an unexpected reward for a measuring change, including steps that allow a second user to provide the unexpected reward, according to an example embodiment;

FIG. 4A is a block flow diagram of a more particular method for providing a user an unexpected reward for a measuring change, including steps for validating performance data, according to an example embodiment;

FIG. 4B is a block flow diagram of a more particular method for providing a user an unexpected reward for a measured change, including validating performance data and steps that allow a second user to provide the unexpected reward, according to an example embodiment;

FIG. 4C is a block flow diagram of a method for applying a first function to determine a performance indicator according to an example embodiment, the performance indicator being used in a method for providing a user an unexpected reward for a measured change, according to an example embodiment;

FIG. 4D is a block flow diagram of a method for sending an unexpected reward, where the unexpected reward is provided by a second user, according to an example embodiment;

FIG. 5A is a schematic showing data being used to train a neural network;

FIG. 5B is a schematic diagram showing performance data being used to predict a communication that is more likely to cause the user to satisfy the performance indicator, according to an example embodiment;

FIG. 6A is an example embodiment of an unexpected reward communication as depicted on a user's computing device, according to an example embodiment;

FIG. 6B is an example embodiment of a loss aversion communication as depicted on a user's mobile device, according to an example embodiment;

FIG. 6C is an example embodiment of a SMS communication as depicted on a user's mobile device requesting training data, according to an example embodiment;

FIG. 6D is an example embodiment of an interface as depicted on a user's mobile device requesting training data, according to an example embodiment;

FIG. 6E is an example embodiment of an interface as depicted on a user's mobile device requesting performance data, according to an example embodiment;

FIG. 6F is an example embodiment of an SMS message as depicted on a user's mobile device requesting performance data, according to an example embodiment;

FIG. 6G is an example embodiment of a graphical user interface as depicted on a user's computing device for a second user to select a performance indicator, according to an example embodiment;

FIG. 6H is an example embodiment of a graphical user interface as depicted on a user's computing device for a second user to provide a response to a poll on social media related to a performance indicator, according to an example embodiment;

FIG. 6I is an example embodiment of a graphical user interface as depicted on a user's computing device for another user to provide a response communication as to whether to provide a reward to the first user, according to an example embodiment;

FIG. 6J is an example embodiment of a SMS message for another user to provide a response to a communication send to the user, according to an example embodiment;

FIG. 6K is an example embodiment of a SMS message for the user to provide a response to a communication send to the user to validate performance data, according to an example embodiment;

FIG. 6L is an example embodiment of a graphical user interface for the user to provide a response to a communication send to the user to validate performance data, according to an example embodiment;

FIG. 6M is an example embodiment a graphical user interface as depicted on a user's computing device for a third user to adjust the magnitude and frequency of unexpected rewards, according to an example embodiment;

FIG. 6N is embodiment a graphical user interface as depicted on a user's computing device displaying goal request and goal data input by user to adjust the goal or a performance indicator for first users to achieve, according to an example embodiment;

FIG. 6O is embodiment a graphical user interface as depicted on a user's computing device for a third user to input a rate to send an unexpected reward message or to send a request to second user to provide an unexpected reward message from the second user, according to an example embodiment;

FIG. 6P is embodiment a SMS message as depicted on a user's computing device displaying a goal request and goal data input by user, according to an example embodiment;

FIG. 6Q is an embodiment a reward communication sent to the first user on a user's computing device, according to an example embodiment; and,

FIG. 6R is an example embodiment a graphical user interface as depicted on a user's computing device for a third user or second user to input a magnitude of an unexpected reward message, according to an example embodiment;

FIG. 6S is an example embodiment a graphical user interface as depicted on a user's computing device for a third user or second user to input a monetary value and a value to produce a reward magnitude that is proportionally related to the difference between the performance data and the performance indicator or goal, according to an example embodiment;

FIG. 6T is an example embodiment a graphical user interface as depicted on a user's computing device for a third user or second user to input a reward frequency or request frequency that is proportionally related to the difference between the performance data and the performance indicator or goal, according to an example embodiment;

FIG. 6U is a SMS message depicted on a user's computing device for a first user to input an attribute that he or she wants to improve, according to an example embodiment;

FIG. 6V is a graphical user interface as depicted on a user's computing device for a first user to select an attribute that he or she wants to improve, according to an example embodiment;

FIG. 6W is a graphical user interface displayed on the social media account of a second user displaying a request to provide a reward and a response to said request, according to an example embodiment; and,

FIG. 7 is a block diagram of a system including an example computing device and other computing devices, according to an example embodiment.

### DETAILED DESCRIPTION

The following detailed description refers to the accompanying drawings. Whenever possible, the same reference numbers are used in the drawings and the following description to refer to the same or similar elements. While disclosed embodiments may be described, modifications, adaptations, and other implementations are possible. For example, substitutions, additions, or modifications may be made to the elements illustrated in the drawings, and the methods described herein may be modified by substituting reordering or adding additional stages or components to the disclosed methods and systems. Accordingly, the following detailed description does not limit the disclosed embodiments. Instead, the proper scope of the disclosed embodiments is defined by the appended claims.

The disclosed embodiments improve upon the problems with the prior art by providing a system that combines clinical medicine, behavioral psychology, and health economics, towards an approach that incentivizes the prevention of weight gain as well as other positive changes using unexpected rewards. The positive changes may include improved blood glucose level; blood pressure; BMI; body weight; calories consumed; heart rate; step count; total sleep; mindfulness; reproductive health; basal body temperature; sexual activities; menstrual flow; intermenstrual bleedings; and, ovulation tests, awake time, deep/light/REM sleep, distance, duration, a sleep pattern, a fat percentage, a muscle mass change, and a change in reading habits, etc. Moreover, chronic diseases, such as diabetes, kidney disease, and heart failure that are frequently caused by ones' own behavior, may also be improved using the methods and systems described herein.

The disclosed embodiments improve over the prior art using systems and methods that provide unexpected reward messages and/or loss aversion messages to cause desired behavioral changes. The disclosed embodiments also improve over the prior art by providing algorithms that determine and improve the efficacy of the unexpected rewards. Lastly, the present invention also improves over the prior art by providing other users (e.g., friends, family, coworkers) the opportunity to send unexpected reward messages and/or loss aversion messages to a user for improved behavior. Receiving the unexpected reward messages and loss aversion messages may provide for increased motivation for changing the behavior. The unexpected rewards that are provided using the methods and systems described herein also improve upon the prior art by providing rewards in a manner that is iterative and not predictable by the user, thus optimally providing the conditions for behavioral change to take place.

The unexpected reward communications implemented in the methods described herein are awarded to a user based on a measured change. The measured change is, for instance, a measurable metric that quantifies the user's behavior. In this way, the provider of the unexpected reward communication can aid a user in changing their behavior in the short-term and long-term. By way of example, the health of a user that is overweight may benefit, among many other things, from regular exercise. In accordance with one or more embodiments of the present invention, the overweight user may measure their exercise activity (e.g., using a smart watch, such as an activity band). After performing their exercise activity, a separate party (e.g., an insurance company, an employer, a sponsor, a friend, a social network) may reward the overweight user with an unexpected reward. Receiving the unexpected reward may encourage the user to exercise more often, and perhaps at higher intensities, longer durations, etc. In this regard, the unexpected reward may be, for instance, a monetary reward such as a gift card, or a verbal reward such as an encouraging message, among others. Regardless, because the overweight user is rewarded for their new behavior (i.e., exercising in this example), the overweight user is more likely to more regularly exercise and therefore improve their health. In this way, users of the disclosed systems and methods may improve upon one or more measurable metrics in relation to their everyday behavior. In other words, the systems and methods described herein may influence a user to demonstrate new, beneficial, measurable behavior.

The described methods and systems additionally allow for enterprise solutions to changing the behavior of individuals associated with the enterprise. For instance, using the systems and methods described herein a health insurance company may improve the health of their customers and reduce costs to their customers. This may be done in a plurality of ways using the described methods and systems. For instance, the health insurance company may provide rewards to users based on consistent improvement in exercise, diet, adherence to medical regimens, among many others. In a similar way, employers may seek to improve the health of their employers by employing the same methods and systems.

In another beneficial aspect of the systems and methods described herein is the ability to obtain measurements and provide rewards asynchronously. For instance, in a clinical trial, medical health questionnaires are often used to obtain data. The medical health questionnaires are typically given on a daily or weekly basis to intelligently measure patient reported outcomes. Using the systems and methods described herein, a portion or all of the medical health questionnaire may be provided to the user at any time of the day. There is also a significant reduction in the latency in acquiring the data. In this way, additional data that may be important in clinical trials (e.g., variation in symptoms throughout the day) may be obtained that otherwise would be too cumbersome to obtain. Moreover, clinical trial patients can be immediately rewarded for their participation in the study. The systems and methods may dramatically increase the participation in the study as an additional benefit of the invention. The rewards provided by the systems and methods described herein may also improve the recruitment in the clinical trial and reduce the attrition of clinical trial patients throughout the study. These benefits, however, are not exclusive to the described embodiment of clinical studies.

i. Operating Environment/System

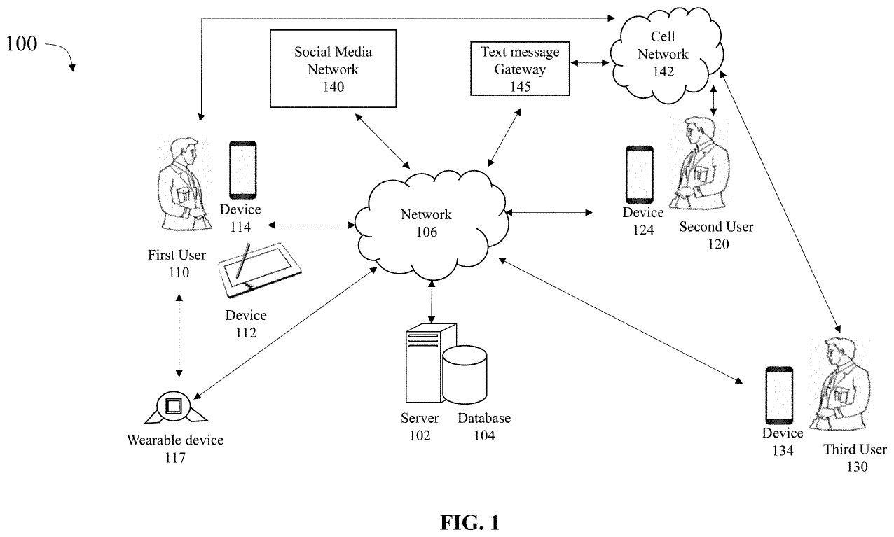

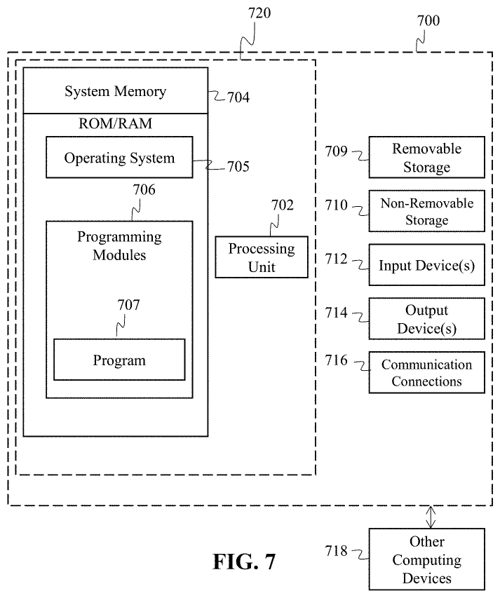

Referring now to FIG. 1, a diagram of an operating environment or system 100 for providing a user an unexpected reward communication for a measured change according to an example embodiment is shown. As illustrated, the system 100 includes a first user 110, a second user 120, and a third user 130. Each user (110, 120, 130) has a device (112, 114, 117, 124, 134) associated with the user. Devices (112, 114, 117, 124, 134) may comprise any computing devices, such as integrated circuits, printed circuit boards, processors, ASICs, PCBs, cellular telephones, smart phones, tablet computers, laptops, and game consoles, for example. Furthermore, the first user 110 may have a wearable device 117, such as a smart watch or activity band (e.g., FITBIT®). A more detailed description of devices is discussed below in relation to FIG. 7.

The devices (112, 114, 117, 124, 134) may be connected to a communications network 106. The communications network 106 may include one or more packet switched networks, such as the Internet, or any local area networks, wide area networks, enterprise private networks, cellular networks, phone networks, mobile communications networks, or any combination of the above. The term “first user” used throughout this application refers to the person for which a measured change is desired. The “second user” used throughout this application may be a person that knows or is associated with the first user 110. For instance, the second user 120 may be a person of trust, such as one or more of a friend, a friend on social media, an acquaintance, a colleague, a coworker, a partner, etc. The term “third user” used throughout this application may be a person and/or entity that may want to help the first user have achieve a measured change. In one embodiment, the third user may be a person who has a vested interest in the first user having the measure change. For example, the third user, may be an insurance company that covers the first user with an insurance policy, an employee of the first user, a sponsor of the first user, a family member of the first user, and/or a friend of the first user, among others.

The network 106 may also be connected to at least one server 102 and at least one database 104. Server 102 includes a software engine that delivers applications, data, program code and other information to the connected devices (112, 114, 117, 124, 134). The software engine of server 102 may perform other processes such as transferring multimedia data in a stream of packets that are interpreted and rendered by a software application as the packets arrive. The database 104 may be a relational database comprising a Structured Query Language (SQL) database stored in a SQL server or a database that adheres to the NoSQL paradigm. Devices (112, 114, 117, 124, 134) may also each include one or more databases.

The network 106 may also be connected to a social media network 140, the social media network 140 being connected to a plurality of the servers, databases, and users via their respective computing devices. The social media network 140 may be one or more of commonly used social networks such as INSTAGRAM®, SNAPCHAT®, FACEBOOK®, LINKEDIN®, STRAVA®, TWITTER®, among others. As will be discussed in greater detail below, the reward communications may be sent via third user(s) using a social network.

As noted above, the network 106 may include cellular networks. Furthermore, the network 106 may be connected to one or more additional cellular networks 142. The cellular network(s) 142 may be used to transmit messages to second users, third users, etc. The cellular network may include a text message gateway 145, or the text message gateway may be a separate entity, such as a third-party entity that provides text messaging services (including both SMS and MMS messages) to server 102. The text message gateway 145 and devices (112, 114, 124, 134) are coupled with cellular network 142 (in addition to network 106). Cellular network 142 may be a mobile phone network comprising a wireless communications network of transceivers.

In certain embodiments, server 102 transmits a request to text message gateway 145, wherein the request includes a generated message and the receiving user's telephone number. Alternatively, server 102 transmits a request to social media network 140, wherein the request includes a generated message and the receiving user's social media network identifier (e.g., a handle, a user ID). Alternatively, server 102 transmits a request to an email server (not specifically illustrated), wherein the request includes a generated message and the receiving user's email address. In response, the social media network 140, text message gateway 145, or email server may send an acknowledgement of receipt of the request back to server 102.

The text message gateway 145 may receive requests and may proceed to send the requested text message to device of the user. For example, in operation the gateway may send a text message to a user's device via cellular network 142 or network 106. Alternatively, the social media network 140 receives the request and proceeds to send the requested message to the device of user via network 106. Alternatively, the email server (not specifically illustrated) receives the request and proceeds to send the requested message (i.e., an email) to the user's device via network 106.

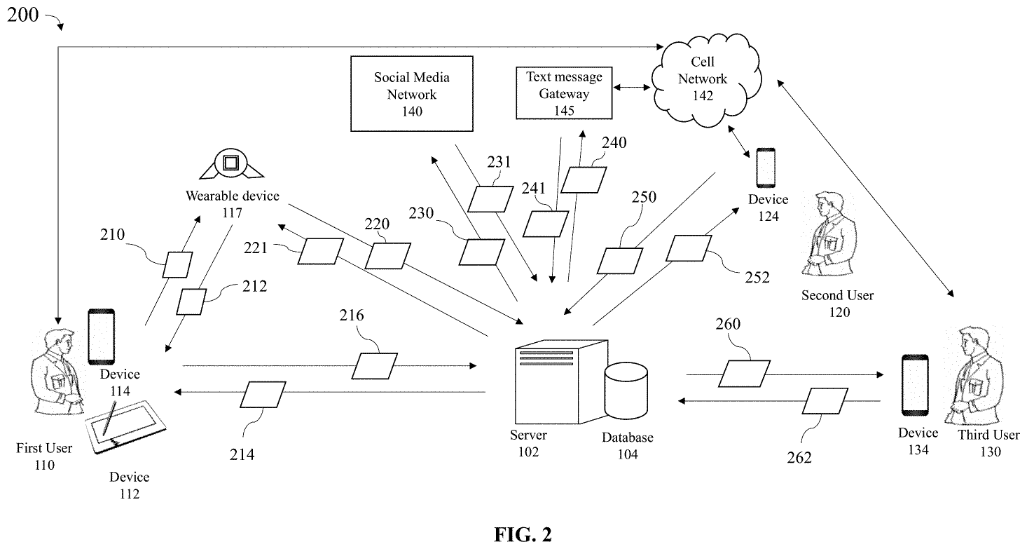

With reference now to FIG. 2, a diagram of an operating environment or system 200 that illustrates the flow of data between the participating entities and components is shown. As illustrated, data (210-262) is transmitted between the devices and other components of the system 100. Data may be provided in a variety of suitable formats, such as one or more of extensible markup language (“XML”), JavaScript object notation (“JSON”), hypertext markup language (“HTML”), cascading style sheets (“CSS”). The data may include both information (e.g., via JavaScript objects, XML), and the information may be displayed according to accompanying HTML and CSS. It is understood that in certain embodiments the data sent between be sent via an appropriate protocol, such as the Hypertext Transfer Protocol (“HTTP”) or Hypertext Transfer Protocol Secure (“HTTPS”).

For instance, data 210 may be sent from the first user's device (112 and/or 114) to the wearable device 117 and data 212 may be received by the first user's device(s) (112, 114) from the from the wearable device 117. Data 216 may be transmitted from first user's device (112 and/or 114) to the server 102 and stored in the database 104. Similarly, data 214 may be transmitted from the server 102 to the first user computing device(s) (112, 114). Data 220 may be transmitted from the wearable device 117 to the server 102, and data 221 from server 102 may flow, in the opposite direction, to wearable device 117.

Data 250 may be sent from the second user's device 124 to server 102, and data 252 may be sent by the server 102 to the second user device 124. Data 262 may be sent from the third user's device 134 and data 260 may be sent by the server 102 to the second user device. Data 230 may be sent to the social media network 140 and data 231 may be sent from the social media network 140 to the server 102. Data 240 be sent to the text message gateway 145 by the server 102, and further to the cellular network 142. Cellular network 142 may send data, in response to data 240, to the text message gateway 145. Data 241 may be sent from the text message gateway 145 to the server 102.

Data 216 sent by the first user's device(s) (112 and/or 114) and wearable device 117 may be stored in a user record, i.e., a first user record. User data that may be included in the first user record may include user identifying information such as name, email address, cell phone number, social media handle, first user contacts (e.g., derived from contact data on the user's phone), permissions of the first user, user training data having metrics associated with attributes of the first user, algorithms, performance data metrics received from the first user or via the wearable device 117, performance indicators having metrics associated with attributes of the first user, goals, messages and communications sent to and from the first user, reward messages, loss aversion messages, unexpected reward messages, loss aversion messages, inquiry messages, and user response messages. However, it is understood that other information may also be included in the first user's record.

Data 214 may include information for displaying reward messages, information for displaying loss aversions messages, requests for training data, requests for performance data, request for permission from the first user to access social media network(s), request for information related to the second user, and displaying communications. However, it is understood that other information may also be included in data 214 transmitted to the first user device(s) (112 and/or 114).

The wearable device 117 may include devices that can be worn on the user's person. The wearable device 117 may include hardware for reading and storing a plurality of data associated with the user (e.g., within in a certain time period). By way of example, performance data (e.g., biometric data such as physical activity data) may be tracked and stored on wearable device 117. The data may be obtained via one or more sensors inside the wearable device. For instance, smart watches such as activity bands (e.g., FITBIT®) may measure the first user's daily steps, mileage, heart rate, blood pressure, body temperature, among others. Such physical activity data may include data relating to one or more of a number of calories burned, the type of physical activity, the length of time dedicated to the physical activity, the intensity of the physical activity, the amount of steps walked, the amount of sleep, the amount of internet of things (“IOT”) device usage, the user's pulse, the user's blood pressure, among others. Such information may be transmitted via radio frequency or vendor application to a reception module in/on the wearable device 117.

Data 210 transmitted to wearable device 117 may include requests for data or input from the first user's device(s) (112 and/or 114). However, other types of data may also be used in or within the spirit and scope of the present invention.

Data 212 may be sent from the wearable device 117 to the first user's device (112 and/or 114) and the server 102 and database 104. Data 220 transmitted directly to the server 102 from the wearable device 117 may include data similar to the same data 216 transmitted by the first user device 114, 112 to the server 102. However, other types of data may be transmitted to and from wearable device 117 and such data is within the spirit and scope of the present invention.

Data 221 may include requests for certain data as well as communications for the first user 110 to read and displayed on the wearable device 117. Data 221 may also include requests from the server 102 to provide information, including biometric data (e.g., data related to physical activity) and other types of data. Other types of data may also be requested by server 102 and are within the spirit and scope of the present invention.

Data 230 may include requests for data transmitted to the social media network. Such data may include requests for input from second users, requests for contact information from the social media network, request for the social media network related to responses from second users, request for contact data to the social media network, data related to gateways and APIs between the social media network and mobile devices, data related permissions, and audio/visual content. However other types of data transmitted to the social media network from the server may also be used and are within the spirit and scope of the present invention.

Data 231 may include requests for data transmitted from the social media network 140 to server 102. Such data 231 may include responses to requests for input from second users, responses to requests for contact information from the social media network, response to requests for contact data to the social media network, data related to gateways and APIs between the social media network and mobile devices, data related permissions, and audio/video content. However, other types of data transmitted from the social media network to the server may also be used and are within the spirit and scope of the present invention.

Data 240 may include requests for data transmitted to the text message gateway and cell network. Such data may include requests for input from users, requests for contact information from users, requests, and messages the users, requests for contact information for second users, data related to gateways and APIs between the social media network and mobile devices, data related permissions, audio video content etc. However, other types of data transmitted to the text message gateway and the server may also be used and are within the spirit and scope of the present invention.

Data 241 may include requests for data transmitted from the text message gateway and cell network. Such data may include responses to requests for input from users, responses to requests for contact information from users, responses from the users, responses related to request for contact information for second users, data related to gateways and APIs between the social media network and mobile devices, data related permissions, audio video content etc. However, other types of data transmitted from the text message gateway and to the server may also be used and are within the spirit and scope of the present invention.

Data 250 may include information that may be stored in the second user record. The information may be stored in the attached database. Furthermore, at least some of the information may be stored in the first user's record. For instance, user identifying information may be included, such as one or more of name, email address, social media handle, cell phone number, second user contacts (from phone), permissions of the second user, user training data having metrics associated with attributes of the second user, algorithms, data for displaying reward messages, information for displaying loss aversions messages, requests for training data, requests for performance data, requested for permission from the second user to access social media network(s), requested for information related to the second user, displayed communications, data related to providing reward messages or loss aversion messages, data related to input on social media feeds, such as likes, dislikes, comments, hashtags, audio/visual content, permissions from the second user, data related to goals for the user, and data related to performance indicators for the first user. However, it is understood that other information may also be included in data 250 transmitted from the second user device.

Data 252 may include information for displaying reward messages, information for displaying loss aversions messages, requests for training data, requests for performance data, request for permission from the first user to access social media networks, request for information related to the second user, graphical interfaces for displaying communications, data related to a request for providing a reward, responses to a request for providing a reward, etc. However, it is understood that other information may also be included in data 252 transmitted to the first user device. All of the data transmitted from the second user device and data transmitted to the second user device may be stored in the second user record that is created for each of the plurality of second users.

Data 260 may include information for displaying reward messages, information for displaying loss aversions messages, information for requests for training data, information to requests for performance data, information related to requests for permission and response from the third user to access social media network(s), information related to requests for information from the third user, information for displaying communications, data related to a request for providing a reward, information related to a request for providing a reward, etc. However, it is understood that other information may also be included in data 260 transmitted to the third user device.

Data 262 may include information that may be stored in the third user record. Furthermore, at least some of the information may be stored in the first user's record. Information, such as user identifying information may be included. User identifying information may include one or more of name, email address, cell phone number, third user and second user contacts (e.g., obtained from the user's device), permissions of the third user, algorithms, data for displaying reward messages, information for displaying loss aversions messages, information input by the third user related to responses to requests for input from the third user, responses to permission from the third user to access social media networks associated with the third user, information requested related to the third user, graphical interfaces for displaying communications, data related to providing reward messages or loss aversion messages, data related to input on social media feeds, such as likes, dislikes, comments, hashtags, audio/visual content, permissions from the third user, data related to goals for the first user to accomplish, data related to performance indicators for the first user to satisfy, data related to the frequency, magnitude and other metrics associated with content to be provided to the first user device. However, it is understood that other information may also be included in data 262 transmitted from the third user device.

As described in greater detail below, a third user record may be created for each of a plurality of third users by server 102, and each third user record may be stored in database 104. All of the data transmitted to the second user device and the data transmitted to the third user device may be stored in the third user record. As described below, second and third users may be prompted to provide the first user with an unexpected reward. The reward may be monetary in nature, and it is understood that the second and/or third user's records may also include credit card information, banking information, and/or other financial information for paying for unexpected rewards.

ii. Methods

As noted above, the systems and methods described herein may influence a user to demonstrate new, beneficial, measurable behavior. As will be described in greater detail below, the methods generally include steps that measure the user's behavior provided via performance data, then the performance data is compared to a performance indicator. If the performance data satisfies the performance indicator, the user is provided with an unexpected reward communication. Moreover, if the user's performance data does not satisfy the performance indicator, the user may be sent a loss aversion communication. Both unexpected reward communications and loss aversion communications may be specifically tailored to encourage (e.g., incentivize) the first user to change their behavior (i.e., provide performance data that satisfies a performance indicator).

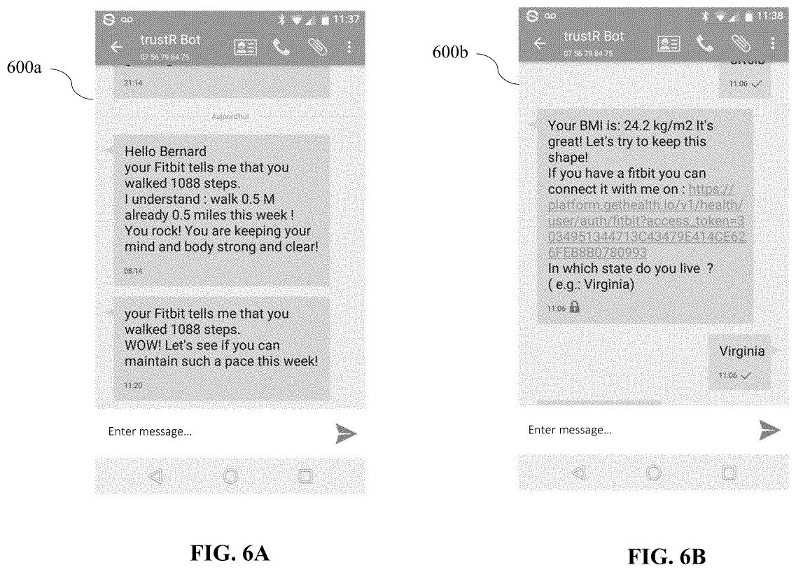

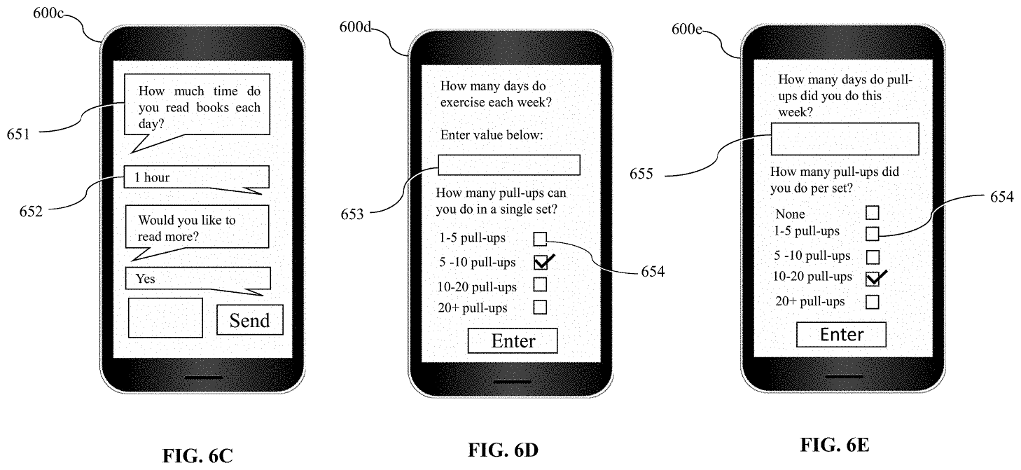

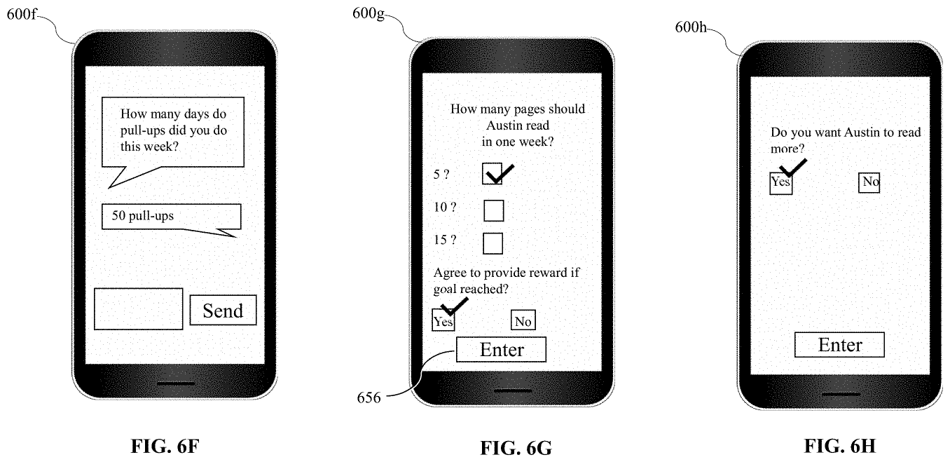

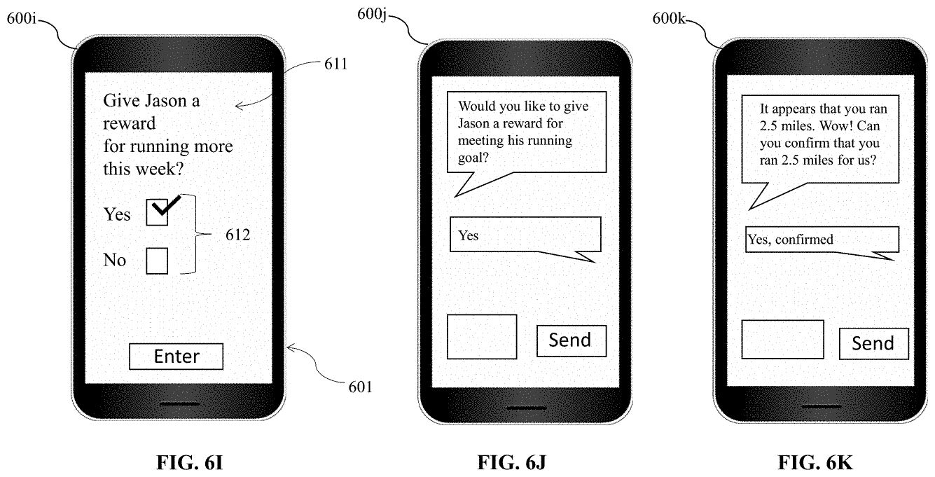

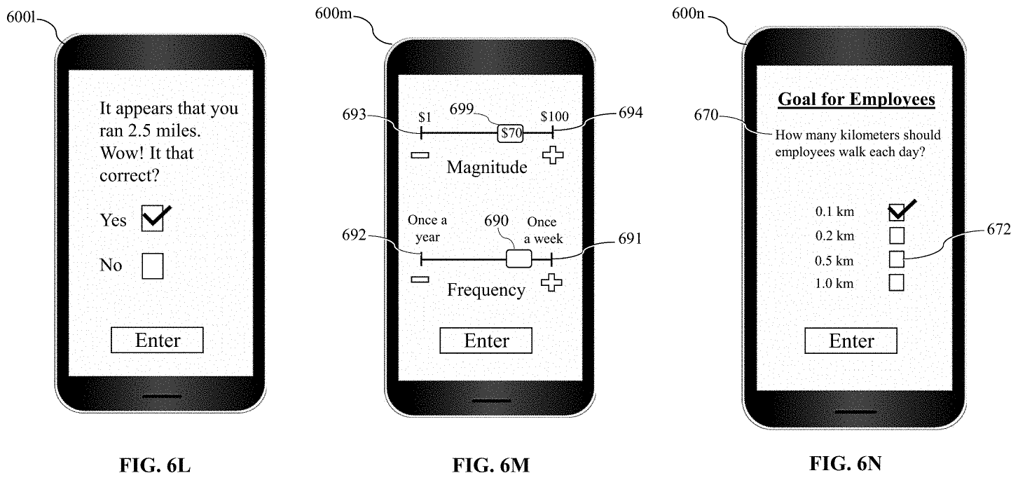

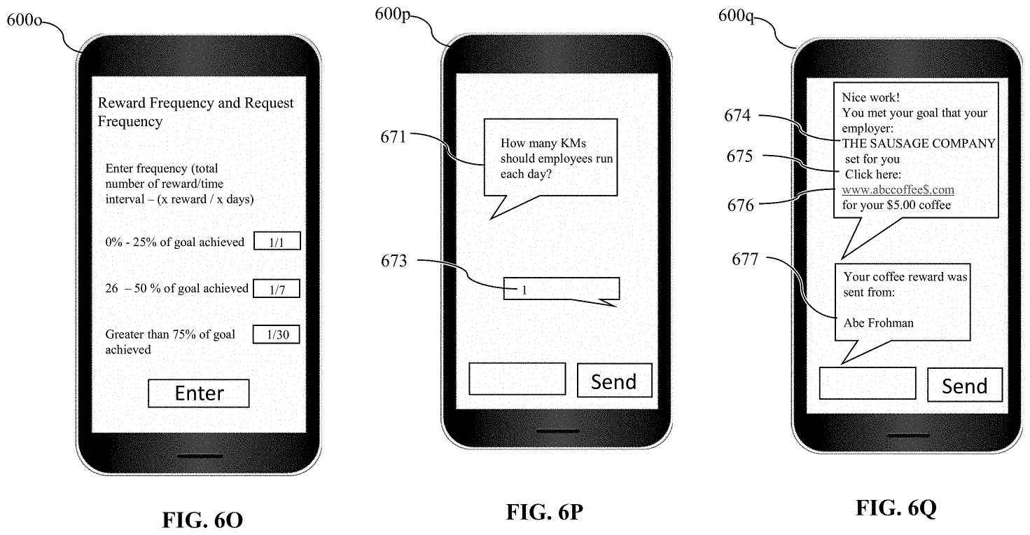

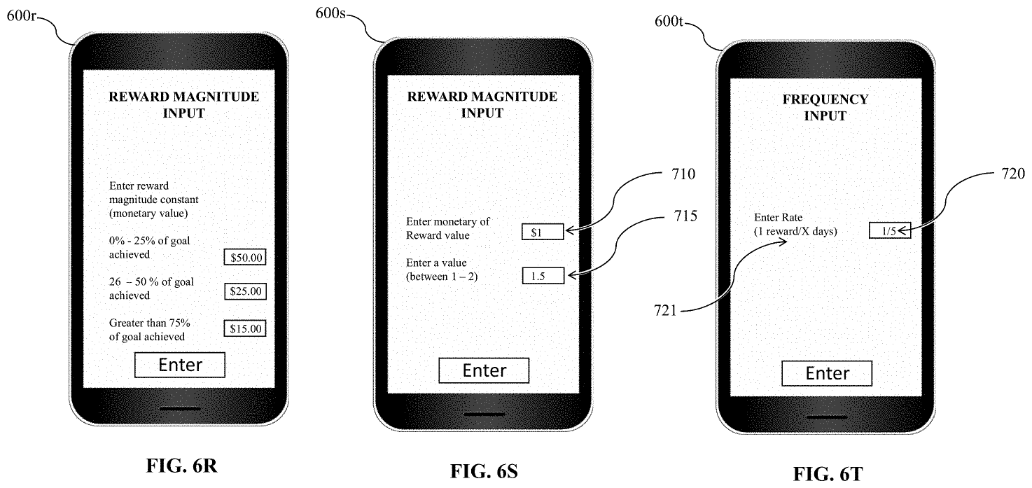

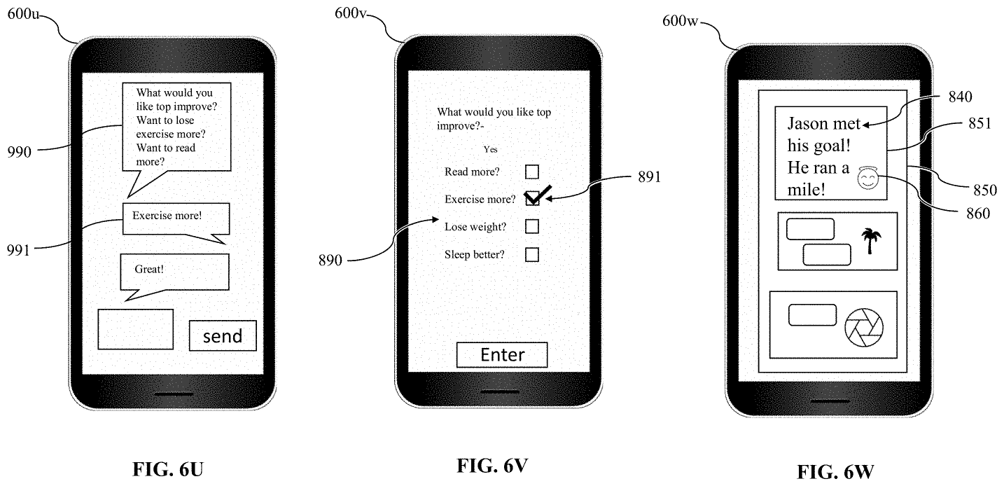

A reward communication is generally intended to influence the measurable behavior of the user. An unexpected reward may include, for instance, monetary rewards and encouraging messages. Monetary rewards generally have some sort of monetary value in the public marketplace. Examples of monetary rewards include digital coupons, gift certificates, money (e.g., USD), among others. The monetary rewards can be specifically tailored to the user based on the particular user's interests. Encouraging messages are generally tailored to a particular user's accomplishments with respect to satisfying the performance indicator. For instance, as shown in FIG. 6A, an unexpected reward communication 600a comprising an encouraging message is shown. The encouraging message includes the performance data (1088 steps), the performance indicator (0.5 miles in a week), and congratulatory phrases (“You rock! You are keeping your mind and body strong and clear!,” and “WOW! Let's see if you can maintain such a pace this week!”). Providing unexpected rewards in this manner may provide an incentive for the user to change their behavior in a measurable way.

A loss aversion communication also is generally intended to influence the measurable behavior of the user. The loss aversion communications may include substantially similar rewards, such as monetary rewards and encouraging messages. However, a loss aversion message may be provided to the user if the performance data does not satisfy the performance indicator. In this way, a loss aversion message may be used to keep a user engaged and to provide further encouragement to satisfy the performance indicator. Loss aversion messages may have lower monetary rewards than their unexpected reward communication counterparts. Furthermore, loss aversion messages may be absent of a reward. Rather, loss aversion messages may remind a user of their performance and performance goals. For instance, as shown in FIG. 6B, a loss aversion communication 600b is shown. As illustrated, the loss aversion message 600b includes a congratulatory phrase (“Your BMI is: 24.2 kg/m2 It's great!” Moreover, the loss aversion message 600b includes a phrase intended to keep the user engaged (“Let's try to keep this shape!”).

As noted above, the use of unexpected reward communications and loss aversion communications is generally intended to influence the measurable behavior of the user. As will become evident with the following description, a plurality of methods may be used to provide communications that influence the first user's behavior. The various steps that may be implemented in the methods described herein are described in greater detail below.

a. User Registration

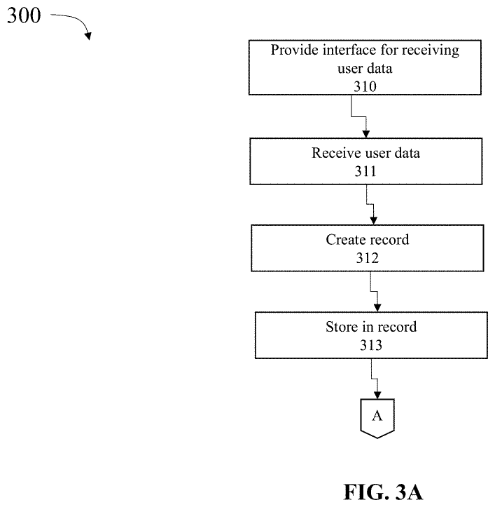

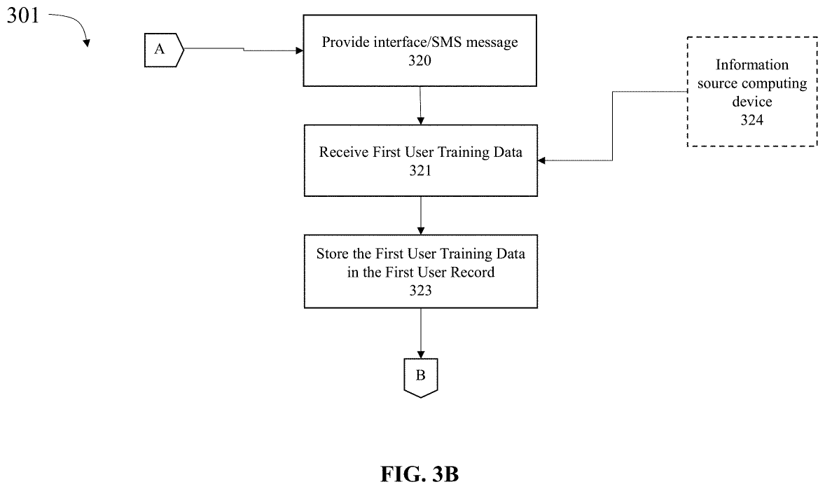

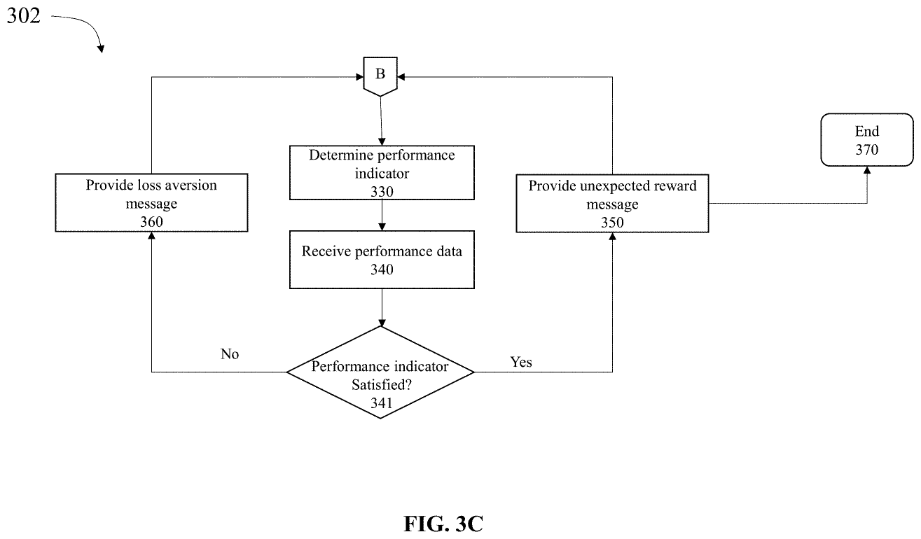

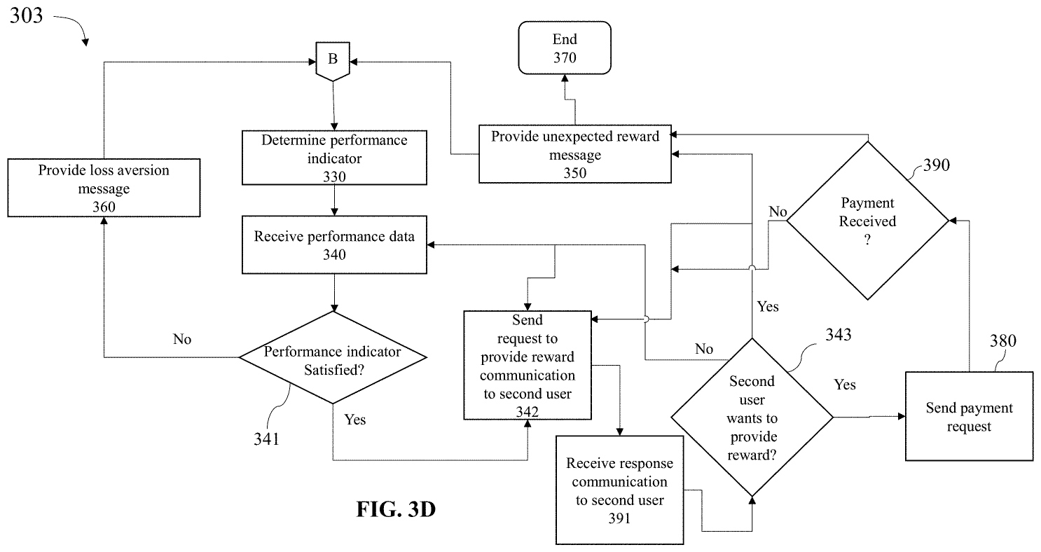

Now with reference to FIGS. 1, and 3A, the methods described herein may utilize a user registration process for any of the users (e.g., first, second, third user(s)). An embodiment of a method 300 for registering a user is shown by FIG. 3A. As illustrated, the method 300 comprises, in step 310, first includes providing an interface for receiving user data for user registration. The user interface for receiving user data may be a variety of different interfaces from which the user may provide data. For example, the processor may be configured for sending a graphical user interface to be displayed on the device of the user. The graphical user interface may include a request for data (e.g., performance data) and/or other data relating to performance indicators (described in greater detail below). The graphical user interfaces may be provided over via a website or HTTP protocol or HTTS protocol. In other embodiments, the processor may be configured for sending a message to a gateway 145 to provide the information via a cellular network 142 to be displayed on the user's mobile device. However, other methods may be used for providing the interface for the user to input data for registration and are within the spirit and scope of the invention.

After the user supplies their user data, next in step 311, the aforementioned server 102 may receive the user data that is transmitted from the user's device. In other embodiments, the system may be configured for sending a message to the cellular network 142 that uses the text message gateway 142 in order to provide a response message to the server. After receiving the data in step 311, the server 102 may then, in step 312, create a record for the user and store, in step 313, the record. The record may be stored in the attached database 104 and in one or more of the first user record, second user record, and third user record. Moreover, a first user record may include all of, or a portion of, second user record(s) and/or third user record(s).

The user may be able to register indirectly using an authentication method provided by an online software application (e.g., a social media network). For instance, the user may register using the GOOGLE® OAuth 2.0 authentication application programing interface (“API”), or a FACEOOK® authentication API, among other APIs.

As the first user continues to use the systems and methods described herein, their data may be continually updated in the first user record. For instance, the first user record may include previous performance indicators, whether or not the performance indicators were met, and any conditions associated with meeting the performance indicator. This data can be used to train a machine learning algorithm. The machine learning algorithm may be used to predictively prepare communications that are most likely to cause the user to change their behavior (e.g., satisfy a performance indicator), as discussed in greater detail below.

b. Establishing a Baseline

With reference now to FIGS. 1 and 3B, the methods described herein include methods for receiving training data having metrics associated with the attributes of the first user to establish a baseline (e.g., baseline performance) for a first user 301. The training data may comprise metrics associated with an attribute of the first user.

The training data generally comprises metrics associated with an attribute of the first user. For instance, the training data may include days, weeks, months, or years of data concerning metrics of attributes measured by the user. A variety of metrics associated with an attribute of the first user may be used. Non-limiting examples of metrics of attributes that may be associated with a health measurement, a labor measurement, an athletic measurement, a scholastic measurement, a biological measurement, and an identifying status. The training data is generally data that has been measured in the past and can be used to set a benchmark (e.g., a performance indicator) for the first user to hit to receive an unexpected reward. Conversely, as will be discussed in greater detail below, performance data is generally current data (e.g., provided in real time). In simpler terms, training data provides the system with previous behavior data, the performance data provides the system with current behavior data, and the measured change is calculated based on the difference(s) between the performance data and training data.

For example, most smartphones include a pedometer that tracks the user's steps. Continuing with the overweight individual example provided above, it might be beneficial to the overweight individual's health to increase the number of steps they take every day. The pedometer training data for the overweight individual may be sent from their smartphone or other wearable device and the pedometer training data may be used to determine a performance indicator. Nonetheless, after receiving the first user's training data (from any of the data packets sent from the first computing device, second computing device, third computing device or wearable device), the method may comprise creating a first user record for the first user, and then storing the training data in the first user record of the attached database. As mentioned above, the first user record will include first user data and may include personal identifying data about the user as mentioned above. For instance, the first user record may include identifying data such as age, gender, geographical location, weight, height, among others. Moreover, the first user record may include training data for a certain metric, algorithms applied, performance indicators etc.

In certain embodiments, the measured change that a user may be provided an unexpected reward for may be, for example, one or more of a sleep pattern, a fat percentage, a muscle mass change, a change in reading habits, adherence to exercise regimens, adherence to therapy regimens, adherence to medication regimens, a respiratory change, or any other sustained change in behavior or biological variable captured by the system, which are associated with the attributes of the user.

Non-limiting examples of attributes that may be used include of a health-related attribute (such as weight or body fat), a labor attribute (such as work efficiency or grammatical errors made at work), an athletic attribute (such as amount of pull ups or steps walked), a scholastic measurement (pages read, better grades achieved, or score received on an examination), and a biological measurement, among others. However, a variety of different attributes may be improved and are within the scope of the present invention. The messages or information to be displayed that may be preprogrammed to provide an adequate response from the user so that the responses include information and metrics associated with attributes. In sum, the received data may be used to establish a baseline for the first user that the first user may improve upon.

The term, “health measurement” refers to a measurement made in relation to a user's health attribute. Non-limiting examples of health measurements that may be made include calorie intake, step count, sleep measurements, adherence to a medication regimen, mindfulness (e.g., time spent meditating), confirmation of having received a particular vaccine (e.g., a seasonal flu vaccine, a COVID vaccine), and contact tracing. Examples of sleep measurements include a time of waking, a time of resting (e.g., falling asleep), a total amount of time slept, a time spent in a one of a deep sleeping state, a light sleeping state, and a REM sleeping state.

In one embodiment, a health measurement is a confirmation of having received a vaccine. Performance indicators that may be used for such a health measurement may include, for instance, compliance with health guidelines for receiving vaccines (e.g., dose, frequency, etc.). In one embodiment, the vaccine is a COVID-19 vaccine.

In one embodiment, a health measurement is a measurement of the individual's participation in a contact tracing effort. For instance, individuals may have a performance indicator to respond to contact tracing efforts, including improved timing and accurate responses (e.g., accurately sharing who the individual has been in contact with).

In one embodiment, a health measurement is a measurement of an individual's participation in sharing their health history. For instance, a performance indicator for providing timely and accurate health history information may be used.

The term, “labor measurement” refers to a measurement made in relation to a user's participation in labor activity attributes. Non-limiting examples of a labor measurement include time sheet accuracy (e.g., number of hours worked, days worked, sick days, paid time off days), medical coding integrity (e.g., determining if a healthcare worker bills work and consumables according to designated standards), compliance to governmental regulation(s), and cyberhygiene.

In one embodiment, the cyberhygiene of an individual is measured. Cyber-attacks are very common in enterprise and approximately 90% of cyber breaches are due to human error. In one embodiment, an individual's susceptibility to phishing attacks is measured. The susceptibility may be measured, for instance, by sending fake phishing attacks to the individual's email inbox and determining if the individual has an appropriate response to the fake phishing attack. For instance, a performance indicator for a cyberhygiene measurement may include determining whether or not the individual clicks the phishing attack email, determining whether or not the individual informs others of the phishing attack, and/or determining whether or not the individual correctly reports the phishing attack to their respective management. Additional performance indicators may be, for instance, the length of time between changing passwords, among others.

In one embodiment, the compliance to governmental regulation(s) of an individual is measured. For instance, in one embodiment, the compliance to GDPR is measured. The compliance to GDPR may include performance indicators such as determining if the individual is sending GDPR-protected information in their email, phone messages, or other.

The term, “athletic measurement” refers to a measurement made in relation to a user's participation in an athletic activity attribute. Non-limiting examples of athletic measurements include a step count (e.g., total number of steps per day), total distance (e.g., distance ran, distance biked), and a duration of exercising.

The term, “scholastic measurement” refers to a measurement made in relation to a user's participation in scholastic activity attributes. Non-limiting examples of scholastic measurement includes a number of pages read (e.g., pages read per day, pages read in a single sitting) and scholastic performance (e.g., a test grade, a class grade, class attendance).

The term, “biological measurement” refers to a measurement made in relation to a user's biology attributes. A biological measurement differs from a health measurement in that the biological measurement is typically an intrinsic property, whereas health measurements are typically extrinsic measurements made in relation to a particular activity. Non-limiting examples of biological measurements include blood glucose, blood pressure, body weight, body mass index, heart rate, and basal body temperature. Measurements may also be made to a user's reproductive health, for instance the user's menstrual cycle (if applicable) may be measured (e.g., menstrual flow, ovulation test, intermenstrual bleeding).

The term, “identifying status” refers to an identity attribute associated with the user. Non-limiting examples of an identifying status include sexual orientation (e.g., LGBTQ, straight), gender identification (e.g., male, female), and veteran status (e.g., honorably discharged veteran, disabled veteran, etc.).

As one example, FIG. 6C shows a text message chat 600c illustrated on a mobile device that may be sent to a user device. As illustrated, the text message chat 600c includes an SMS message 651 that may be transmitted to the user computing device via the cellular network and gateway. The user may respond with a response message 652 to provide the requested training data.

As another example, FIG. 6D is an example of an interface 600d as shown on a mobile device that may be provided for the user. As illustrated, the interface 600d includes a form element 653 for the user to enter a numerical value. Furthermore, the interface 600d includes a plurality of input fields 654 (illustrated as checkboxes) that are responsive to the user for selecting the appropriate training data. FIGS. 6C and 6D illustrate exemplary ways to obtain training data from a user. The figures are illustrated in relation to the first user. However, similar messages and interfaces may be produced for the second and/or third user(s).

In one embodiment, server 102 transmits a request (as in data packet 240) to text message gateway 145, wherein the request includes the message generated by the server and includes the user's telephone number. The request and response receive may be sent and received, respectively, via HTTP or HTTPS. In some embodiments, server 102 transmits a request to a social media network 140, wherein the request includes the message generated by the server and the consumer's social network handle. In some embodiments, server 102 transmits a request to an email server, wherein the request includes the message generated and the consumer's email address. In response, the social media network 140 or text message gateway 145 or email server sends an acknowledgement of receipt of the request back to server 102. The text message gateway 145 receives the request and proceeds to send the requested text message (as shown in FIG. 6C) to the user's device using the user's cellular telephone number. For example, gateway 145 may send a text message to device via cellular network 142 or network 106. Alternatively, the social media network 140 receives the request and proceeds to send the requested message to device of the user via network 106. Alternatively, the email server receives the request and proceeds to send the requested message (i.e., an email) to device of user via network 106. It is understood that the messages and interfaces and responses transmitted between the server and each of the users described in this application may be done in a similar fashion. However, other embodiments may be used and are within the spirit and scope of the present invention.

The messages and information to be displayed may preprogrammed (e.g., by server 102) based on information provided by any of the first user, the second user, and/or the third user. It is also understood that the interfaces and messages for receiving training data may be provided to any of the first user, second user, and/or third user. For example, if the third user is the first user's employer, then the third user may select to improve the labor measurement of the first user. In another case, if the third user is the first user's parent, then the third user may select that the third user would like the first user to improve scholastically. In other cases, the second users may be one or more of the first user's close friends on a social media network. The second users may select one of a plurality of attributes of the first user that the second user's want to see the first user improve.

After providing the interface step 320, method 301 includes receiving the data in step 321. In step 321, the training data input is transmitted over communications network 106 to the server 102 and received by the server 102. It is understood that the training data may be one of the data packets that is transmitted over the communications network to the server (as illustrated in FIG. 2). Alternatively, or after providing the interface in step 320, the method 301 includes providing training data from a computing device (e.g., information source computing device) in step 324. The information source computing device may be one of the first user computing device, the second user computing device(s), the third user computing device(s), and a wearable device.

In other embodiments the system may be configured to send messages to a cellular network to send a SMS message to the first user so that the first user may provide a response message with training information. The information computing device may be the first user's computing device, or any other appropriate computing device that sends training data to the server. In one embodiment, the information source computing device comprises one of the first user computing device, the second user computing device, the third user computing device, a wearable device processor, and a sensor processor associated with a sensor. In other embodiments the information computing device may be a processor or device associated with a cellular network that provides the training information received after receiving a response by SMS message from the first user. In one non-limiting embodiment, a human resource manager (i.e., a third user) may include training data the first user via the human resource manager's computing device.

c. Performance Data, Performance Data Validation, and Performance Indicators

As noted above, the communication that is sent to the first user (unexpected reward, loss aversion) may be chosen based on the performance data, and if the performance data satisfies a particular performance indicator. The performance indicator may include one or more criteria (e.g., goals) for the user to satisfy. Continuing with the overweight individual example described above, a performance indicator relating to an exercise attribute might be a total distance ran. In such an example, the performance indicator criteria might be a total distance ran of at least 0.5 miles. Additional criteria, such as the total distance ran per day, or per week, or per month, may be used to define the performance indicator. Regardless, the performance indicator is generally a single measurable criterion or a set of measurable criteria by which the performance data is evaluated.

In one embodiment, the system may provide to one of the users an interface for inputting information for users to provide a performance indicator that the first user may want to achieve. For example, FIG. 6G illustrates a graphical user interface 600g for a user (other than the first user) to input content. The content may be alphanumeric or may be selected from a plurality of options by the user (as illustrated). In FIG. 6G, the graphical user interface 600g may be provided to a plurality of second users in the network to provide information related to the performance indicator. The information populated in the graphical user interface 600g may correspond to attribute of the first user that the first user intends to improve. For example, if the first user wants to read more, and the first user currently does not read any pages per week, then the system may be configured to apply a function to the training data received in order to generate an interface configured to receive alphanumeric data that will provide a goal for the first user to achieve. In the embodiment illustrated in FIG. 6G, the first user is not an avid reader (5 pages per week), so the system is configured such that the server provides to the second users an interface to select the number of pages that the first user (Austin) should read. Additionally, the interface 600g also provides an area 656 on the interface for the user to enter/submit their preferences for providing a reward to the first user.

It is understood that other embodiments may also be used for calculating the performance indicator. For example, the system may be configured for receiving data from the plurality of different second users and apply a function to the data received in order to calculate a performance indicator. For example, FIG. 6H illustrates a user interface 600h to be provided to a plurality of second users via the social media feed for the second users to select if the second user may want the first user wants to improve upon an attribute. After receiving response(s) from a plurality of second users, the processor may aggregate the response data to determine a performance indicator. Other functions or algorithms may be simple algorithms such as improving the amount of the training data by a certain percentage. For example, if the training data indicates that the user reads a certain or average number of pages per week, then performance indicator may be calculated by an algorithm such as PI=P*(1+X), where PI is the performance indicator, P is the number of pages per week read and Xis a certain percentage. In another example, if the training data indicates that the user wants to lose weight, and the user consumes 5000 calories per day, then the algorithm to calculate the performance indicator may include an algorithm such as PI=C*(1−Z), where PI is the performance indicator, C is the number of calories consumed per day and Z is a percentage. Other algorithms, such as non-linearly applied functions, may be used or in or within the spirit and scope of the present invention.

The performance data may be self-reported, provided automatically by the first user's technology (e.g., smart watch), or provided by another party. Regardless, the performance data includes measured metrics that can be evaluated relative to the performance indicator.

With specific reference now to FIG. 3C, a method 302 for providing an unexpected reward for a measured change in a user is shown. As illustrated, the method, starting from off-page connector B (following the user registration), and in step 330, the method includes determining a performance indicator. In this regard, the performance indicator is generally for the metrics (measured data) associated with the attribute of the first user. After determining the performance indicator in step 330 (for example according to one embodiment described above), the method includes step 340 of receiving performance data. The performance data may be sent by first user computing device, second user computing device, third user computing device or wearable device 117 or the information source computing device (described above)) or any of the first computing device, second computing device or third computing device. Moreover, the performance data may comprise the metrics associated with the attribute of the first user. Similar to the other training data received, the performance data may be input by the first user into the first user computing device via a first user computing device interface.

With momentary reference now to FIG. 6E, an example interface 600e that may be used to be provided to the first user computing device is shown. The interface may be sent over the communications network and may be configured for receiving data such as performance data. As illustrated, the interface in FIG. 6E illustrates areas 655 where the user may enter a numerical value associated with the performance data of the attribute of the first user. Area 654 of the interface illustrates another embodiment of how the second user may select an alpha numerical value (or range of values) to indicate information related to the performance data. The illustrated interface, or an SMS-message equivalent may also be sent over a cellular network. Thus, in some embodiments the server may also be configured for (i) sending a message to a cellular network via a text message gateway in order to display a text message and (ii) for receiving a response message from the text message gateway to receive the performance data. Additionally, the system is also configured for receiving their performance data via the wearable device 117 directly or via the first computing device, which may be one of the data packets 216 or 220.

With reference back to FIG. 3C, after receiving the performance data, in step 341, if the performance indicator is satisfied, then the method may move to step 350, to provide an unexpected reward message to the first user. Conversely, if the performance indicator is not satisfied, then the process moves to step 360, and the method may include providing a loss aversion message. The method may be repeated (starting again from off-page connector B). If, for instance, the user did satisfy the performance indicator and receive an unexpected reward message in the prior iteration, the performance indicator may be recalculated. For instance, continuing with the overweight user example, if the performance indicator were to run 0.5 miles in a day, and on a first day the user runs 0.6 miles and receives an unexpected reward, the performance indicator may be adjusted to 0.6 miles the next day. The performance indicator may be adjusted as desired to encourage the user to change their behavior. As will be described in greater detail below, machine learning may be used to automate and optimally calculate the performance indicator and/or the magnitude, frequency, and type of rewards that are most likely to encourage the user to produce the measured change (i.e., change their behavior).

With reference now to FIG. 3D, a more particular method 303 for providing an unexpected reward for a measured change in a user is shown. In the illustrated embodiment of the method 303, the method includes additional steps that enable a second user to provide the unexpected reward communication. As noted above, the system/operating environment 100 may include a second user. The second user may be a friend, family member, co-worker, among others. Regardless, the second user may have a personal connection with the first user and may want to encourage the first user to change their behavior. Continuing with the overweight user example, the overweight user may have a friend that is concerned with their weight and wants them to lose weight to improve their health and life. The friend (i.e., second user) can register using the registration steps described above in relation to FIG. 3A. Following registration, the friend can opt-in to provide rewards for the overweight user and may also participate in setting the performance indicator and/or determining if the performance indicator has been satisfied. The registration steps may generally provide the system with second user data, such as a second user identifier (e.g., a name, a nickname).

In one embodiment, a method comprises (i) sending, over the communications network, to a second user computing device of one or more second users, a request to provide a reward to the first user if the validated performance data satisfies the performance indicator, (ii) receiving, over the communications network, from the at least one second user computing device, at least one response communication to the request to provide the reward, where the at least one response communication comprises a second user identifier and an approval communication, (iii) selecting, from the at least one response communications, a provider of the unexpected award communication, where the provider is one of the second users, (iv) sending, over the communications network, to the first user computing device, the unexpected reward communication, where the unexpected reward communication includes a second user identifier of the provider of the unexpected award communication.

Similar to the method described above in relation to FIG. 3C, the method includes, in step 330, determining a performance indicator as described above. After step 330, the method 303 moves to receiving step 340. Receiving step 340 includes receiving the performance data, similar to the embodiments described above. Next, the processor, in step 341, determines whether or not the performance indicator has been satisfied in step 341. The processor may be configured to determine if the performance data satisfies the performance indicator by comparing the data associated with the received performance data to the performance indicator.

If the performance indicator has been satisfied, then in step 342, a request to provide a communication is sent (e.g., via a server) over the communications network to the second user including a message asking if the second user wants to provide a reward (see e.g., FIGS. 6G-6J). The message sent to the second user may include a graphical user interface, the graphical user interface including text notifying the second user of the first user's accomplishment(s) and a request for the second user to provide the unexpected reward. In other embodiments, the second user may have already agreed to provide a reward if the first user's performance data satisfies the first user performance data indicator (as illustrated in FIG. 6G).

After the second user inputs a response (either via SMS message or user interface) a response communication or message (including the approval communication or denial communication) is send to and received by the server (at step 391). An approval communication includes data that indicates the user wants to provide a reward message to the first user. A denial communication includes data that indicates the user does not want to provide a reward message to the first user. If a denial communication is received, then the service may send a loss aversion message to the first user. The response communication or message is stored in the first user record and may also be stored in the record associated with user that sent the communication, which may be used for further processing (e.g., training machine-learned algorithms).

If the user wants to provide the reward at step 343, the graphical user interface may provide the second user with reward options (e.g., monetary rewards, words of encouragement) using the graphical user interface. As an example, FIG. 6I shows a graphical user interface 600i as shown on a mobile device. The interface 600i includes a request for a reward 611, and options for the user 612 to respond. In other embodiments, the system may be configured for providing a message to the text message gateway in order for the cellular network to send an SMS message to the second user to respond affirmatively or negatively respond. An example embodiment of such an SMS message 600j provided by the cellular network after receiving a message from the processor is included in FIG. 6J. If the second user wants to provide a reward, the determination is made based on the response communication received from the messages sent by the second user computing devices.

After the second user responds to the communication with an approval for providing the unexpected reward, in step 350, the first user is provided with the unexpected reward message provided by the second user. Alternatively, at step 343, if the second user does not want to provide an unexpected reward, then the server may receive a denial communication in response to the request sent to the second user. The denial communication generally indicates that the second user does not want to provide a reward and the method may move back to step 340. Alternatively, the process may move back to step 342 to send other second users request to provide reward communications (or messages) (as described above) and/or interfaces. The interfaces may be used to by the second user may send a denial or approval response communication. In an alternative embodiment, if the second user does want to provide a reward, the process may also move back to step 342 and send subsequent requests to provide reward communications (or messages) to other second users. In other words, the process may sequentially inquire from other second users until one of the second users provides a reward to be sent to the first user.

Provided that the second user, or a plurality of second users provides a reward at step 343, the unexpected reward communication that is sent to the first user may include a second user identifier. For instance, the second user identifier may be a name, social media handle, or other identifier. The first user may feel more encouraged that the second user has taken a personal interest in the first user's personal improvement. Thus, the unexpected reward communication sent by the second user may provide the first user with additional motivation to continue to satisfy the performance indicator over time, thereby changing their behavior. It is understood that this second user identifier may be a component of the data that is transmitted from the second user devices to the server directly or via the cellular network and text message gateway. Additionally, in other embodiments, where second users want to provide an unexpected reward, the processor may be configured to perform a random selection of which second user identifier may be included in the unexpected reward message. Having second users decide when the first user should receive an unexpected reward communication or message, or a loss aversion message may also be used to further randomize and make the reward even further unexpected.

The reward communication sent to the first user may include an actual reward, such as a monetary reward. FIG. 6Q illustrates the reward communication 600q and includes a link to receive the reward for a product that was provided by the second user. The link may be associated with the reward magnitude (M) that was calculated and that is proportionally related to a difference between the performance data and the goal, wherein a magnitude constant for calculating the reward magnitude is provided by at least one of the second user computing device and the third user computing device. The reward communication may also include the second user's information (such as a second user's name, address, work information, phone number) or other information associated with the second user that is provided in the second user record and associated with the second user identifier so that the first user knows who provided the reward. The reward communication 600q illustrated in FIG. 6Q may be provided according to a reward frequency that is proportionally related to a difference between the performance data received and the goal, wherein a frequency constant for calculating the reward frequency is provided by at least one of the second user computing device and the third user computing device. In one embodiment, the reward communication 600q illustrated in FIG. 6Q is based on the goal data for the goal of the first user that is provided by at least one of the second user computing device and the third user computing device. The message may also include a user identifier 674 associated with the user that provided the goal data. The message may also include a calculated reward magnitude 676. The message may also display a graphical representation of the second user identifier 677 of the second user that provided the reward. The message may also include a link to a web location that where the first user may redeem the unexpected reward. It is understood that other embodiments may also be used and displayed and are within the spirit and scope of the present invention.

In one embodiment, after receiving a response from the second user that the second user wants to provide a reward at step 343, the method 303 may include sending a payment request step 380. The payment request may be sent to the second user in order to prove provide payment for the unexpected reward. In other embodiments, the system may be configured for automatically debiting the unexpected reward payment from the second user's account using stored payment data found in the second user's record. Next, the method may include determine if the payment has been received at step 390. Provided payment has been received, the method 303 includes providing the unexpected reward message in step 350.

In one embodiment, a method comprises (i) sending, over the communications network, to a second user computing device of one or more second users, a request to provide a reward to the first user if the validated performance data satisfies the performance indicator, (ii) receiving, over the communications network, from the at least one second user computing device, at least one response communication to the request to provide the reward, where the at least one response communication comprises a second user identifier and an approval communication, (iii) selecting, from the at least one response communications, a provider of the unexpected award communication, where the provider is one of the second users, and (iv) sending, over the communications network, to the first user computing device, the unexpected reward communication, where the unexpected reward communication includes a second user identifier of the provider of the unexpected award communication.

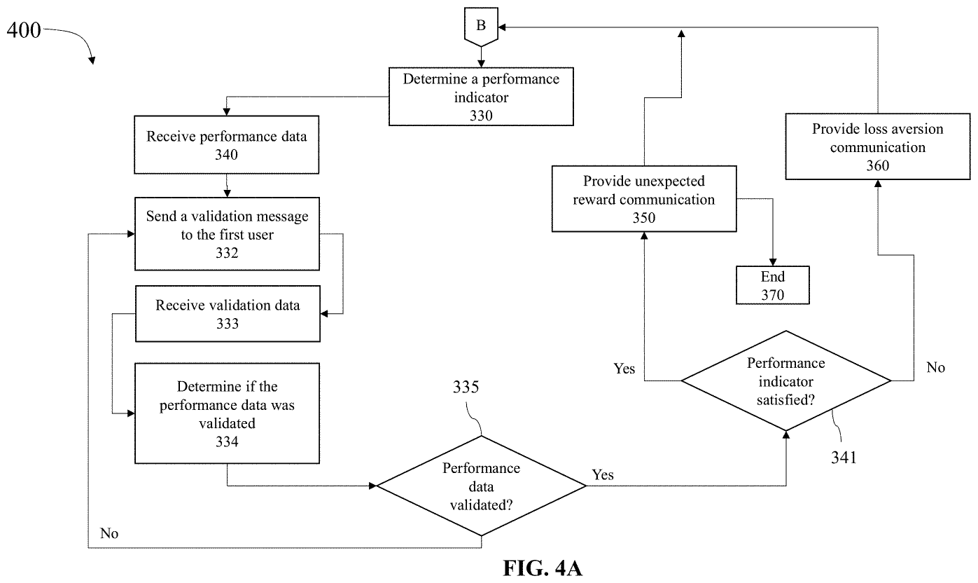

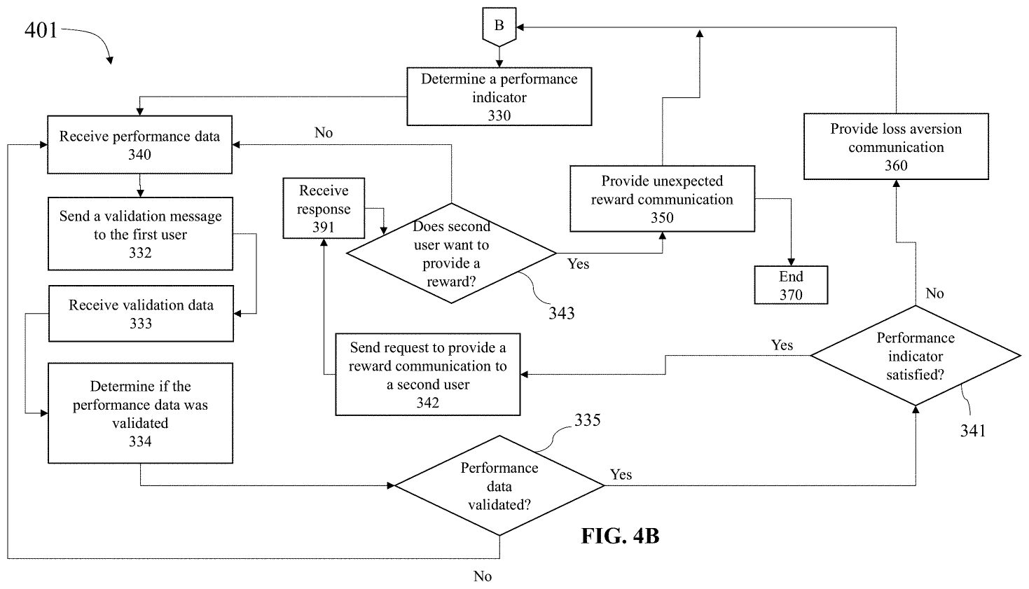

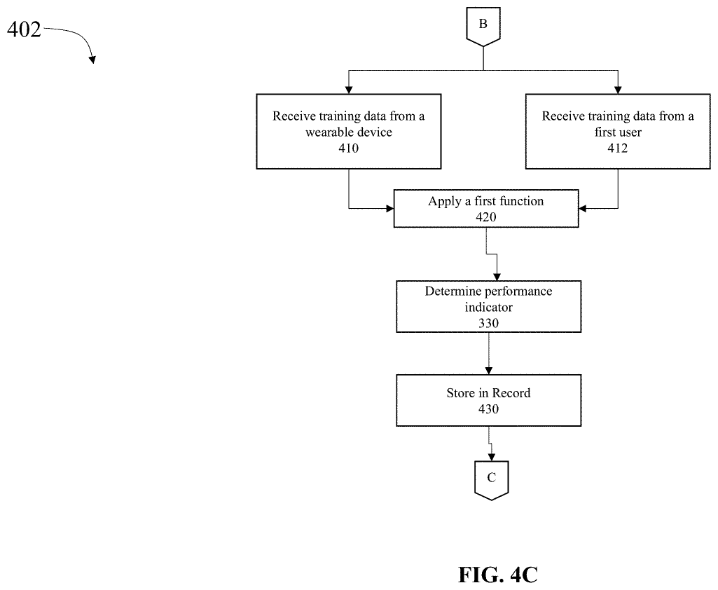

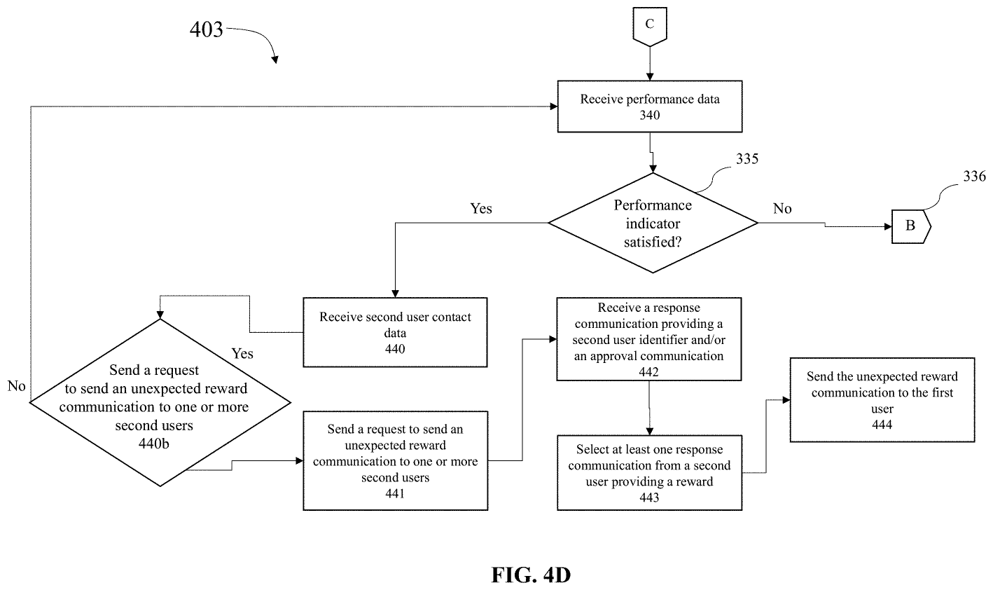

With reference now to FIG. 4A, a method 400 may comprise validation steps (332-335) to validate the performance data. Any suitable method for validating the data may be used. For instance, the data may be validated via a computer algorithm, or directly via confirmation by the first user, among others. Validating the performance data may be necessary to ensure that the first user is rewarded for actually satisfying the performance indicator.

Continuing with the overweight individual example, the overweight individual might track their running session using their smart watch (wearable device 117). The smart watch might track an unrelated activity, such as driving in a car, riding a bus, or riding a bike, and the smart watch might report this data to the server, identifying it as performance data associated with a running session. In this regard, the performance data may be identified as false performance data and excluded from being considered as satisfying a performance indicator. Alternatively, the performance data may be validated, and the steps described for providing an unexpected reward may be executed.

Following off-page connector B (after user registration in FIG. 3B), the method 400 includes in step 330 determining a performance indicator and receiving the first user's performance data step 340. As mentioned above, the performance data may be included in the data packets 220 from the wearable device 117 and/or received from the first user computing device 112 in data 216. The method may additionally include, in step 332, sending a validation message to the first user to request validation data. This validation message may be included in the data 221 to the wearable device and/or data 214 to the first computing devices. Depending on the measured metric, the system may evaluate the performance data to determine if the received first user performance data has been validated in step 334. Similar to receiving performance data and training data, the processor may be configured for sending a user interface to the first user computing device, or other computing devices, to have a user input information. In this way, the user may also directly validate the performance data (described below). If the performance data is validated at step 335, then the process move to step 341 to determine if the performance indicator has been satisfied and communication steps (350-360) may be executed. If the performance data is not validated at step 335, then the process may move to step 332 and continue the process and steps 333-335 are repeated.

As mentioned above, the validation data may be provided by the first user directly. For instance, the first user may receive a message, which may be included in the data 221 to the wearable device and/or data 214 to the first computing devices, and requests the first user to validate the performance data. Continuing with the overweight individual example, the overweight individual might be sent a message (such as text message chat 600k illustrated in FIG. 6K) (e.g., a link to a custom web page, an SMS message sent via the cellular network and gateway) stating, “It appears that you ran 2.5 miles. Wow! Can you confirm that you ran 2.5 miles for us?” In other embodiments, the message may a graphical user interface (such as illustrated in graphical user interface 600l in FIG. 6L) that is provided to the first user device. In certain embodiments, the messages to be displayed as user interfaces may be sent via HTTP or HPPS. As shown in FIG. 6L, the user interface 600l may include radio buttons to select yes or no, along with a submit/enter button. If the message is sent by SMS message, the first user might respond by “submitting” the response includes sending a “yes” or “no” SMS message. However, other messages and interfaces may be used and are within the spirit and scope of the present invention. Thus, in one embodiment, a method includes providing, over the communications network, to at least one of the first user computing device, the second user computing device and the third user computing device, a first interface for receiving validation data.

In another aspect, the processor may be configured to include text certain recognition software and programmatically request an additional response if the first response does not seem responsive to the question posed to the user.

Certain criteria, or algorithms may be used to validate the performance data. In this way, the validation process might include determining if any measured metrics are inconsistent with expected values. For instance, performance data might be measured for what is reported to the server as being a running session. To validate the data, the total distance, the user's speed, or other metrics might be evaluated for inconsistencies. As a non-limiting example, the running session might include a metric that is inconsistent within known human abilities to run, such as an average speed of 50 miles per hour. In sum, the validation steps (332-335) may be used to reduce false positives for satisfying the performance indicator, and to ensure that finite, unexpected reward resources are efficiently used.

In one embodiment a method comprises (i) receiving, over the communications network, from a validation computing device, validation data for validating at least a portion of the performance data associated with the attribute, (ii) determining if at least a portion of the performance data was validated using the validation data, where if the portion of the performance data was validated using the validation data, the portion of the performance data is validated performance data, and (iii) determining if the validated performance data satisfies the performance indicator.

Referring now to FIG. 4B, a more particular method 401 for providing a user an unexpected reward for a measured change is shown. As illustrated, the method 401 includes the aforementioned determining a performance indicator in step 330 and receiving a first user's performance data in step 340. Further, the method includes the validation steps (332-335) described above in relation to FIG. 4A. Even further, the method includes the steps described above in relation to FIG. 3D for enabling a second user to provide the unexpected reward (steps 342, 343). Thus, as one may appreciate from the foregoing figures, the aforementioned user registration steps (FIGS. 3A-3B), validation steps (FIGS. 4A-4B), may be combined with the core steps (330, 340).

FIG. 4C illustrates a more particular process of determining a performance indicator. FIG. 4C Illustrates that in step 410 training data may be received from the wearable device as a part of data packet 220. Additionally, or alternatively, in step 412 training data may be received from the first user device as a part of data packet 216. Next, the data received in steps 410 and 412 may be validated using one of the validating processes described above. Next, in step 420 the processor or server may be configured to apply a function from the training data received. The type of function applied to the training data received may depend on the attribute for which the training data is associated with. For example, if the training data is associated an athletic measurement or metric that the user would like to increase, then then performance indicator maybe calculated by an algorithm such as PI=P*(1+X), where PI is the performance indicator, P is the number of minutes it takes for the user to run a certain distance and X is a certain percentage. For example, if the training data is associated an athletic measurement or metric that the user would like to decrease, then then performance indicator maybe calculated by an algorithm such as PI=P*(1−X), where PI is the performance indicator, P is the number of minutes it takes for the user to run a certain distance and X is a certain percentage. In this manner the algorithm allows the user to have a performance indicator that establishes a goal for which the user wants to achieve. However, other types of algorithms may be used and are within the spirit and scope of the present invention.

By way of another example, if the training data indicates that the user wants to lose weight, and the user consumes 5000 calories per day, then the algorithm to calculate he performance indicator may include an algorithm such as PI=C*(1−Z), where PI is the performance indicator, C is the number of calories consumed per day, and Z is a percentage. However other algorithms may be used or in or within the spirit and scope of the present invention.

Next, in step 330, the performance indicator is calculated based upon the algorithm that selected and the attribute that the first user wants to improve. Next, in step 430, the processor stores the performance indicator calculated from the training data after applying the first function into the attached database in the first user record. However, it is understood that the training data may also be included in the second user record and third user record.

In one embodiment, the first user may be sent a text message or user interface requesting the first user to input an attribute that he or she wants to improve. For instance, FIG. 6U is a text message chat 600u depicted on a mobile device, requesting the first user to input an attribute that he or she wants to improve. As another example, FIG. 6V depicts a graphical user interface 600v illustrated on a mobile device for a first user to select an attribute that he or she wants to improve.

The graphical interface 600v illustrated in FIG. 6V may be provided by the server, to the first user computing device, in a manner similar to the other interfaces described herein. After initially registering, based upon the information provided by the user, the server may select certain messages that will be provided to the user computing device. In other embodiments, the server may send a message configured to display a list 890 of attributes that the first user may want to improve. By selecting one of the items in the list at the direction of the first user, then server then may receive, over the communications network (similar to how other messages are sent to the server from the first computing device as described herein), a response that includes the item that the first user selected. Based upon the item that selected, the server may include that data in the first user record. Additionally, after that information has been selected, the server may move to step 330 to determine the performance indicator. In one embodiment, the user may provide may select the particular attribute that the user intends to improve. In one embodiment, the user may select the attribute that it intends to improve by providing input 891. Input 891 may be a gesture, such as a swipe, push, pinch, check, etc., on the user interface (such as interface 600v illustrated in FIG. 6V) as executed on the computing device by the user. It is understood that other attributes and algorithms may preprogrammed into the server so that the list of attributes that the first user may want to improve varies from what is shown or illustrated in FIG. 6V.

Referring to FIG. 6U, a text message chat 600u is shown. In certain embodiments, an SMS message 990 may be provided by the server to the first user computing device in a manner similar to the other interfaces described herein. After initially registering, based upon the information provided by the user, the server may select certain messages that will be provided to the user computing device. The messages may include the first user cellphone number and may be sent to first user's cellphone via the text message gateway. In certain embodiments, the server may send the message configured to display an SMS message having attributes that the first user may want to improve. By sending a response message 991 to the SMS message with one of the items in the list at the direction of the first user, the server then may receive the response message 991. The response message 991 may include the metric that the first user wants to improve upon. Based upon the metric that is selected, the server may include that data in the first user record. Additionally, after that information has been selected, the server may execute step 330 to determine the performance indicator. In one embodiment, the user may select the particular attribute that the user intends to improve. In one embodiment, the user may select the attribute that it intends to improve by providing a response message 991. It is understood that other attributes and algorithms may preprogrammed into the server so that the list of attributes that the first user may want to improve varies from what is shown or illustrated in FIG. 6U.

FIG. 4D Illustrates another process 403 for sending a loss aversion communication or unexpected reward communication to the first user. In step 340, the server receives their performance data from the first user. As mentioned above, the performance data may be included in data packet 216 transmitted from the first user computing device after receiving a response to an SMS message or input provided by the first user in an interface on the first user's computing device (such as in FIG. 6E-6F, respectively), or may be included in data packet 220 from the wearable device. However, other embodiments of receiving the performance data may also be within and scope of the present invention.

Next, in step 335, the processor or server may be configured for determining whether the performance indicator has been satisfied. As explained above, the server may determine if the value of the metric of the performance data received is greater than or equal to the performance indicator metric in order to determine if the performance indicator has been satisfied. In other embodiments, the server may determine if the value of the metric of the performance data received is less than or equal to performance indicator to determine if the performance indicator has been satisfied. For example, if the performance indicator is for the user to read 10 pages a day, and the performance data received from the first user computing device indicates that the user read four (4) pages a day, then the performance indicator has not been satisfied. On the other hand, if the performance data received from the first user computing device indicates that the user read 20 pages in one day, then the performance indicator has been satisfied. Other embodiments of calculating a performance metric may also be used and are within the spirit and scope of the present invention.

As mentioned above, the server is configured to programmatically make the determination based upon the algorithm an attribute of the user for which the user is trying to improve. If the performance indicator has not been satisfied, then the process moves to step 336. On the other hand, if the performance indicator has been satisfied, then the process moves to step 440. In step 440, the processor is configured to receive the second user contact data via data packets 250 from second user computing device. In certain embodiments the second user contact data may already be stored in the first user record. In certain embodiments, the second user contact data may be provided by the first user computing device and may be in a data packet 216 provided by the first user computing device. The second user contact data may also be provided by the first user computing device after the server sends a request, over the communications network to the first user computing device for the second user contact data. In other embodiments, the server may use the first user's permission, social media handle or other contact information in order to send a message over the communications network to the social media network. The message to the second users may be made via the social media network, such as by posting a message to the first user's social media feed in order to solicit a response from at least one or a plurality of second users. In other embodiments, the second user contact or other data related to the second user may be transmitted from the social media network in a data packet 231 over the communications network. After locating the second user contact data to which to send the communication, then the process moves to step 440b to send a request to send an unexpected reward communication to one or mor of the second users.

In step 440b, the server searches the first user record to determine the frequency at which the first user is to receive the unexpected reward or loss aversion message. The term “request frequency” may mean the rate at which the server sends to the second user computing device the request to provide the reward to the first user. The term “reward frequency” may mean the rate at which the server sends to the first user computing device the unexpected reward communication or loss aversion communication. The term “frequency” may be used to refer to both the reward frequency and the request frequency. It is understood that the request frequency and rate frequency may also be used in various combinations to further randomize the unexpected rewards. The server may be further configured for sending to the one or more second users, the request to provide the reward to the first user prior to sending the unexpected reward communication. The request to provide the reward to the first user may be in accordance with a request frequency. In certain embodiments, the request frequency may be proportionally related to a difference between the performance data and the goal, wherein a “frequency constant” for calculating the request frequency is provided by the third user computing device. The server may also be configured to provide an unexpected reward communication to the first user according to a reward frequency that is proportionally related to a difference between the performance data received and the goal the user wants to achieve. The frequency constant for calculating the reward frequency is provided by at least one of the second user computing device and the third user computing device.

To determine the frequency, the server may look up in the attached database the first user record the appropriate reward frequency for the unexpected rewards. Furthermore, the server may look-up in the attached database the appropriate request frequency to send second user's request message for providing an unexpected reward to the first user. In certain embodiments, the request message to provide an unexpected reward message may only be sent to the second user if the first user should receive an unexpected reward (e.g., based on criteria other than the performance indicator). In other embodiments, the request message to provide an unexpected reward message may be sent to the second user after the server determines the performance data satisfies the performance indicator (as explained above). The reward frequency and request frequency, simpler terms, is how often the server sends unexpected reward messages to the first user or request messages to the second user(s) further differentiates the present invention from the prior art in that it further provides a means for sending a randomized unexpected reward.

The reward frequency and request frequency may be associated with frequency data that is stored in the first user record. The reward frequency may be calculated by a machine-learned algorithm or rules-based algorithm. The reward frequency may be calculated based on data present in the first user's record. For instance, the first user's record may include data that indicates the period between rewards that is more like to cause the first user to consistently satisfy their performance indicator(s). Similarly, the request frequency for the second user(s) may be calculated based on data present in the second user's record. For instance, the second user's record may include data that indicates how often the second user is likely to provide unexpected rewards to the first user. For instance, the second user may be more likely to provide a reward on the first user's birthday, or on a holiday. The second user may be more likely to provide a reward on a weekend, or if the first user and second user had communicated recently. Alternatively, the second user may be more likely to provide a reward if the first user satisfies the performance indicator consistently (e.g., every few days).

The reward frequency at which the user should receive a loss aversion communication or unexpected reward communication may be relatively simple, such as once a week, once a month, once a year, etc. However, other types of distributions or predictive algorithms may be used in order to encourage the first user to satisfy the performance indicator. Such algorithms may include a Poisson distribution. The distributions or predictive algorithms may be stored in the first user's record.

The request frequency may also be relatively simply, such as once a week, once a month, once a year etc. However, other types of distributions or predictive algorithms may be used to solicit the second user to provide unexpected rewards. Such algorithms may include a Poisson distribution. The reward frequency and request frequency may be stored in the first user's record and may be accessed from the attached database by the server.

The reward frequency and request frequency may be proportionally related to a difference between the performance data received by the server (via to the first user computing device, cellular network via the text message gateway or social media network) and the goal the first user is trying to achieve (e.g., the performance indicator). In other words, the more difficult the goal is for the first user to achieve, then the greater frequency that the rewards are provided to the first user. Further, the more difficult the goal is for the first user to achieve, the more frequent requests to provide an unexpected reward are sent to the second user. Stated differently, the lower probability that a goal has been met (e.g., performance indicator satisfied), then greater rewards may be provided to the user. On the other hand, the easier the goal is for the first user to achieve, the lower the frequency at which the rewards are be provided or the requests to provide rewards are sent to the second user. Stated differently, the higher probability that a goal that has been met, then the lesser rewards may be provided to the first user. The goal may be the same as the performance indicator calculated by the server or as provided by the third user computing device. However, other embodiments may be used and are within the spirit and scope of the present invention.

A frequency constant for calculating the request frequency and reward frequency may be provided by the third user computing device in a message from the third-party computing device. The message may be included in a data packet 262 provided by the third-party computing device. For example, the frequency constant may be provided in ranges (see for example, interface 600o in FIG. 6O). However, in other embodiments the frequency constant may be a numerical value greater than one. If the frequency constant is between zero and one, the reward frequency of reward weight may be provided in an inversely proportional matter. For example, the rate of rewards may be provided in a directly proportional manner of RF=(D)×K, where RF is the reward frequency, D is the difference between the performance indicator (PI) or goal (G) and the performance data (PD), where K is a constant rate where R is a standard value rate at which rewards are to be sent (1 reward/5 days). Similarly, for example, the rate at which second user receive requests to provide rewards may be provided in a directly proportional manner of: QF=(D)×K, where QF is the request frequency, D is the difference between the performance indicator (PI) or goal (G) and the performance data (PD), where K is a constant rate at which rewards are to be sent (1 reward/5 days). However, it understood that other embodiments or algorithms may be used to send rewards at different frequencies, including at randomized frequencies.

FIG. 6T is an example user interface 600t that may be provided to a user to enter the value of the rate to send messages. The interface 600t includes a display message 721 displayed on the second user interface that was provided by the server in a message that may have been transmitted in a data packet 240 via the text message gateway, in a data packet 230 via the social media network, or in a data packet 260 to the device. It is also understood that the data input by the user may be transmitted in a data packet 241 via the text message gateway to the server, in a data packet 231 via the social media network to the server, or in a data packet 262 from the device. It is understood that the constant rate allows for the reward frequency or request frequency to be adjusted. It is understood that other embodiments may be used and are within the spirit and scope of the present invention.

As another example, FIG. 6N illustrates a graphical user interface 600n for setting a goal for a first user to reach. The interface 600n may be provided to the first user computing device computing device over the communications network and may be included in the data packet 214. In other embodiments, the goal request may be provided to the second user computing device computing device over the communications network and may be included in the data packet 252. In other embodiments, the goal request may be provided to the third user computing device computing device over the communications network and may be included in the data packet 260. It is understood that the information displayed on the computing device for the goal request may be adjusted depending on the user.

The processor may be configured for sending, over the communications network, to at least one of the second user computing device and the third user computing device, a goal request for goal data for the goal of the first user. The processor may also be configured for receiving, over the communications network, from at least one of the second user computing devices and the third user computing device, the goal data for the goal of the first user and storing, in an attached database, the goal data for the first user. The goal data comprises an improvement over the training data.

FIG. 6N illustrates a goal request interface 600n displayed on the interface of either the second user computing device or the third user computing device. The goal request interface 600n includes a request message 670 requesting a goal parameter for employees. As mentioned above, in certain embodiments, the server is configured for and sends, over the communications network, either second user computing device in a data packet 252 or to the third user computing device in a data packet 260, a goal request for goal data for the goal of the first user. However, in other embodiments, the server may be configured for sending the goal request to the first user computing device, second user computing device or to the third user computing device by sending a message in a data packet 240. The goal request may include either the first party phone number, the second party phone number(s) or the third-party phone number(s), respectively to the text message gateway which in turn then sends the message via SMS message to the intended recipient.

An example of an SMS message sent to the second or third user displaying a goal request 671 is illustrated by text message chat 600p shown in FIG. 6P. After the goal request is sent, the server may receive the goal data for the goal of the first user from at least one of the first user, second user computing devices and the third user computing device. The goal data may be input by user on the computing device of either the first user, the second user or the third user by SMS message or a graphical interface. The goal data may be stored in the first user record in the attached database. The goal data may also be stored and associated with the second party record or third-party record, if necessary.

As illustrated by the interface 600n shown FIG. 6N, goal data input 672 may be selected by either the second or third user, which may then be transmitted to the server via data packet 250 (if from the second user) or data packet 262 (if from the third user) over the communications network to the server to be stored in the first user record, or the second user record or third user record (depending on the sender). The text message chat 600p shown in FIG. 6P also illustrates goal data input 673 by either the second or third user, which may then be transmitted to the cellular network and then sent to the server via data packet 241 from text message gateway to be stored in the first user record, or the second user record or third user record (depending on the sender).

It is understood that the goal data comprises information that indicates an improvement over the training data. In certain embodiments, the goal request may be adjusted depending on the training data received from the first user. For instance, the interface or text message provided to the second or third user may compel the second or third user to provide goal input that is more likely to cause the first user computing device to send performance data different than the training data. As illustrated in FIG. 6O, the goal request indicates “How many KM should employees walk each day ?” and the responses include options to select 0.2, 0.5, 0.7 and 1 km. This goal request perhaps may be for first users that are known to have training data reflecting a rather sedentary lifestyle. On the other hand, if the first users were known to have a more active lifestyle, As reflected by the training data, then the options may have been 5 kilometers, 10 kilometers, 15 kilometers, or 20 kilometers.

It is also understood that other embodiments of interfaces may also be used for receiving a value from the third user computing device. In other embodiments, the server may send a message to the text message gateway including the cellular phone number of the third user computing device to then send a corresponding text message or SMS message to the third user computing device corresponding to the cellular phone number in the message sent to the text message gateway. After the information or goal data is received, the goal data may be used to determine the performance indicator of the first users. For example, if the third user is an employer, and the first user(s) are the employees of the employer, then the goal may be stored as the performance indicator in the user record for each of the plurality of first users associated with the third user. If the difference between the goal provided by the third user is, say a unit 10, then the reward frequency that rewards or requests are sent to the first user and second user, respectively, will be at a rate that is more frequent than if the difference between the goal set by the third user is say 0.

In another example, if the employer or third party provides goal data that establishes a goal for the employees to walk or run one kilometer each day, then the employee's performance data that shows the employees walk 0.2 miles per day will receive rewards at a rate less frequent then the employee who runs 10 miles a day. The proportionality of the rewards based on the difference between the performance data and performance indicator may be encouraging for the first users.

While computer algorithms may be used to set these frequencies, it is understood that the third user, such as an employer, may set the request frequency at which the employees are provided rewards. It is also understood that the reward frequency or rate at which the requests to provide an unexpected reward are sent to second users may also be set by the third user.

FIG. 6O is a user interface 600o provided over the communications network to the second or third user(s) to input data associated with the reward frequency rate at which first users should receive a reward. The interface 600o may be provided over the communications network to the third party, or to the second user, to input data associated with the request rate at which second users should receive a request to provide a reward to the first users. For example, the user may establish ranges for the rates at which the rewards are to be provided or the requests are to be send to the second user computing devices.

With continued reference to FIG. 6O, for example, if the difference between the goal provided by the user (e.g., the performance indicator), is between 100-75 on a scale from 0-100 (less than 25% of goal achieved), then the reward will be provided at a rate of 1 reward per day (1 reward/1 day); if the difference between the goal is between 74-50 (26%-50% of goal achieved) on a scale from 0-100, then the reward will be provided at a rate of 1 reward per week (1 reward/7 days); and if the difference between the goal is between less than 25 (greater than 75% of goal achieved) on a scale from 0-100, then the reward will be provided at a rate of 1 reward per month (1 reward/30 days). In this manner the rates at which rewards are provided are proportional to the difference between the performance data and the performance indicator. It is understood that other rates at which reward should be provided or rates at which the server should send a request to send an unexpected reward communication to one or more of the second users may also be used and are within the spirit and scope of the present invention.

In one embodiment a graphical user interface may be provided to the third user computing device via HTTP or HTTPS. Such an example of a graphical user interface for setting the reward frequency or request frequency (frequency constant) is illustrated in FIG. 6E. In the user interface 600m embodiment illustrated in FIG. 6M, the graphical interface includes a slider from which a user may interact with in order to adjust the reward frequency of which the request for providing an unexpected reward messages provided to a second user (or also to adjust the request frequency or rate at which requests to provide rewards are send to the second user). It is understood that in the embodiments where the frequency may be adjusted, the third user may interact with the slider 690 between providing rewards or requests to provide rewards at difference frequencies. For example, the third user may interact or inputs information (such as a gesture, swipe, push, pull, pinch, etc.) in order to have a less frequent reward sent to the first user computing device or a request to provide a reward to the second user computing devices (for further randomization and unexpectedness of when to provide the rewards). In the present embodiment, the slider 690 allows the third user to provide rewards at a constant rate for all users between a reward frequency of once a year at a second end 692 of the scale and on the first end 691 of the scale a more frequent time period, such as once a week. As the slider 690 is moved between the first end 691 and the second end 692 of the scale, the reward frequency is adjusted.

The third user could determine that rewards would be more beneficial to be sent more often to a certain first user. The third user may adjust the reward frequency first users receive rewards more often or to adjust the request frequency to allow the second users to receive request to send an unexpected reward communication to the second user after the system has received data from the first user computing device indicating that the performance data or is greater than the value of the performance indicator. Similarly, the third user could determine that rewards would be more beneficial to the first user to receive reward less frequently and use a gesture to move the slider to the other end of the scale. The reward frequency data and request frequency data received from the third user computing device may be stored in the first user computing device record. It is understood that the reward frequency data and request frequency data may be transmitted over the communications network in data packets 262 from the second computing device but may also be transmitted via the text message gateway 145 in data packet 241 if the input has been received via SMS message.

Referring back to step 440b in FIG. 4D, to determine if a request to send an unexpected reward communication to the second users should be sent, the server may look up in the first user record when last the first user received an unexpected reward. If the reward frequency, which may be defined as (amount of rewards/an interval of time) is greater than or equal to the first user frequency (amount of rewards the first user received/interval of time), then the server may then move to step 441. On the other hand, if the reward frequency, which may be less than or equal to the first user frequency (amount of rewards the first user received/interval of time), then the server may instead not conduct step 441 but move to step 340 and continue to receive performance data.

By way of example, if the third user has set the frequency at which first user is to receive rewards after the performance data received satisfies the performance indicator is set (and stored in the first user record) to allow the system to provide only one (1) reward for every seven days (1/7) and the first user has not received any rewards in the last seven days (0/7), then the server may determine that the reward frequency is greater than the first user frequency and allow the process to move to 441. On the other hand, if the third user has set the frequency to provide only 1 reward for every seven days (1/7) and the first user has received 1 reward in the last seven days (1/7), then the server may determine that the reward frequency is equal to the first user frequency and not allow the process to move to 441 and instead move to step 340.

It is also appreciated that others means of calculating the reward frequency and request frequency are within the spirit and scope of the present invention. For example, the system may use predictive modeling to score the data in the first user record and apply predictive models (e.g., machine-learned models) to determine the best frequency at which to provide a reward based on the data in the first user record. Such predictive modeling may be different distributions including Poisson distributions, negative binomial distribution, however, other types of models may also be used and are within the spirit and scope of the present invention.

FIG. 6M is an example of a graphical user interface 600m that may be provided to the third user to adjust the magnitude of the reward that is provided for each award. The interface 600m includes a slider 699 that may be moved between a first end 693 of the scale and a second end 694 of the scale in order to adjust the monetary amount or magnitude of the reward. For example, in the present embodiment, on a first end 693 the dollar amount of the award to be provided to the user is $1 per reward. On the second end 694 the dollar amount of the award to be provided to the user is $100 per reward. The amount of the reward to be provided to the user may be adjusted by the user manipulating or providing a gesture (Push, pull, pinch, swipe, etc.) on the slider 699 to move the slider from left to right. By moving the slider, the 3rd user may adjust the size of reward to be given to the first user. The position of the slider along the scale or track may indicate or display the monetary amount. For instance, in the illustrated embodiment the monetary amount is $70 per reward based on the position of the slider. After an amount of the reward has been entered via the graphical interface into the third user computing device, the data may be sent via the communications network in one of the data packets 262 sent from the third user computing device to the server. The magnitude data associated with the monetary amount for each reward may be stored in the first user record in the database.

It is also understood that the reward amount may also be provided by the second users. In such cases, the user interface 600m may be provided to the second users over the communications network in data packet 260 or via the text message gateway via data packet 240. The response from the second user related to the monetary reward may be provided via the communications network in a data packet 250 or via text message gateway 145 the data packet 241. The response from the third user relate to the monetary reward may be provided via the communications network in a data packet 260 or via text message gateway 145 the data packet 241.

In one embodiment, an unexpected reward communication comprises a reward magnitude that is proportionally related to a difference between the performance data and the goal (e.g., performance indicator). The magnitude constant for calculating the reward magnitude is provided by at least one of the second user computing device and the third user computing device. In simpler terms, the greater the difference between the goal and the received performance data, then the larger the reward to be sent to the user. For example, if the difference between the goal provided by the user is between 100-75 on a scale from 0-100 (less than 25% of goal achieved), then the reward will be provided at a certain magnitude (M); if the difference between the goal is between 74-50 (26%-50% of goal achieved) on a scale from 0-100, then the reward will be provided at certain magnitude less by an amount (P) (thus M-P); and if the difference between the goal, or in other embodiments the performance indicator, is between less than 25 (greater than 75% of goal achieved) on a scale from 0-100, then the reward will be provided at a certain magnitude less than the magnitude of M-P (thus M-P-Q). In this manner the magnitude at which rewards are provided are proportional to the difference between the performance data and the performance indicator. It is certainly understood that other magnitudes that rewards should be provided may also be used and are within the spirit and scope of the present invention. For instance, it is also understood that the server may also allow users to establish different equations to adjust the magnitude such that the reward magnitude is calculated using a difference between the performance data and the goal.

Algorithms may be used that are different than those embodied herein that allows the user to establish a single constant. For example: M=K(D)×(V), where M is the magnitude (where M may be a monetary value or some other value), K is a constant (numerical value of at least 1) and D is the difference between the performance indicator (PI) or goal (G) and performance data (PD) (D=PI or G-PD) and where V is a value of a reward (which be a monetary value or some other type of unit.) In such a model, the greater the difference (D) between the PI and PD or G, the larger the magnitude of the reward that may be sent to the user. FIG. 6S illustrates a user interface 600s for a user to enter a minimum reward 710, and where the constant 715 is a constant greater than one. The constant 715 will produce reward magnitudes that are directly proportional to the difference between the performance indicator (PI or G) and performance data (PD). It is understood that if a value between 0-1 is used, the reward provided to the user will be inversely proportional to the difference between the performance indicator (PI) and performance data (PD).

In step 441, similar to step 342, the server sends a message or communication over the communications network to the second user computing device a message asking if the second user wants to provide a reward (see e.g., FIGS. 6I and 6J). The message sent to the second user computing device may include a graphical user interface (as illustrated in FIG. 6J), the graphical user interface may include text notifying the second user of the first user's accomplishment(s) and a request for the second user to provide the unexpected reward. In other embodiments, the second user may have already agreed to provide a reward if the first user's performance data satisfies the first user performance data indicator (as illustrated in FIG. 6G). In other embodiments, the communication is sent by SMS message via a text message gateway. In such embodiments, the server sends a message to the text message gateway, thereby connecting with the cellular network and sending an SMS message to the second user computing device. As mentioned above, the message to the text message gateway may include data related to the message and the cellular number of the second user (or third user, depending on the circumstance). The cellular number may be found in the first user record or a plurality of user records as described above. The SMS message sent to the second user (or third user) may include a request that the second user provide a reward to the first user as illustrated in FIG. 6J.

Referring to FIG. 6I, the graphical user interface 600i may provide the second user with reward options (e.g., monetary rewards, words of encouragement) using the graphical user interface. As an example, the interface 600i includes a request for a reward 611, and options for the user 612 to respond. Next, in step 442, the system will receive from either the social media network, second user computing device, or text message gateway a responsive response message or communication providing a second user identifier and/or an approval communication or denial communication. An approval communication may comprise data associated with the input received from the users indicating that the first user should be provided with a reward. This data maybe input after the second user interact with a graphical user interface provided on the second user device (as illustrated in FIG. 6G) via a gesture, push, swipe, pinch, entering other alphanumerical values, etc. Similarly, the approval communication may comprise data associated with the input or SMS message received from users indicating that the first user should be rewarded. This input may be received after the second user inputs data into the remote computing device of the second user indicating that the second user approves of sending a reward message to the first user as illustrated by text message chat 600j in FIG. 6J. A denial communication may comprise data associated with the input received from the users indicating that the first user should not be provided with a reward. This data may be input after the second user interact with a graphical user interface provided on the second user device (as illustrated in FIG. 6G) via a gesture, push, swipe, pinch, entering other alphanumerical values, etc. Similarly, the denial communication may comprise data associated with the input or SMS message received from users indicating that the first user should not be rewarded. This input may be received after the second user inputs data into the remote computing device of the second user indicating that the second user does not approve of sending a reward message to the first user as illustrated by text message chat 600j in FIG. 6J.

This message may be included in the data packet 231 from the social media network, a data packet 250 from the second user device, the data packet 241 from the text message gateway (depending how the response from the second user is sent to the server). It is understood that these response messages from the second user may also be stored in the first user record as well as the second user record and third user record. It is understood the second user identifier is a unique data element associated with the second user and may be stored in the second user record of either the social media network's database or in the cellular network database that identifies the particular second user that responded to the message. A second user identifier associated with the response to the request to provide an unexpected reward from the second user may also be stored in the first user record. The date and time of the response from the second user may also be stored in the first user record. In one embodiment, the approval communication may be a response from the second user via the social media network, text message gateway, or second user computing device that indicates that the second user does not want to provide a reward to the first user. In one embodiment, the denial communication may be a response from the second user via the social media network, text message gateway, or second user computing device that indications that the second user does not want to provide a reward to the first user.

d. Social Media Networks

In certain embodiments, the processor is further configured for, prior to the sending the second user computing device the request to provide the reward, receiving, over the communications network, from the social media network messages and information in response to messages sent from the server. The server may also be configured for sending messages having information that require responses from the social media network 140. This may include messages comprising the second user contact data and may also comprises a second user contact name and at least one of a second user email address and a second user mobile phone number. This information may be stored in a second user record in an attached database, the second user contact data. However, in certain embodiments where privacy is more of a concern, this information may not be sent or maybe sent in an encrypted matter. Additionally, the second user contact data may also be provided from the third user computing device via a data packet 262 sent from the third user in response to a request for information regarding second users sent to the third user computing device 134 in data packet numeral 260.

It is understood that the data packets and data transmitted between the devices used in this application are exemplary and are meant to show how data and messages between the users and server. Each of these data packets or data represented here and may be transmitted in a variety of different ways including via HTTP or HTTPS. The messages that are sent by the server to the social media network may include these social media handles, and other contact information regarding the second users and also associated with the first users. In certain embodiments the message is sent to the social media network compel the social media network servers to post or send messages to the second users that are associated or are included in the message from the server. This step is akin to the step 342 in FIG. 3D.

Such social media messages may include polling questions for the second users to react or interact with. Such polling questions may be included in a social media feed, a direct message to the plurality of different users. The polling questions may include information that indicates if the second user wants to provide a reward to the first user. The response may include data related to a like or dislike, a thumbs up, an interaction with a data element on the feed of the social media network, or other embodiments allowing the second user to indicate that it approves or disapproves of providing the reward to the first user. After the social media network received responses to polling questions Indicating that the second users either approves or disapproves of the first user receiving an unexpected reward, these social media network may transmit in a data packet, such as in data packet 231 to the server, which may be stored in the record of the first user. The server may then calculate based upon an algorithm if a threshold minimum amount of users has approved of providing the unexpected reward to the first user. The approval may be calculated from the responses received from the poll or post that was published on social media. For example, data related to a like, thumbs up, heart, smiley face, or other icon that indicates that the second user approves or affirmatively agrees with the concept of sending a message to an unexpected reward message to the first user. FIG. 6W shows a graphical user interface 600w displayed on the social media account of a second user displaying a request to provide a reward and a response to said request, according to an example embodiment. The interface 600w a social media feed 850 of a second user and a post 851 provided by the social media network based on the message provided by the server. This post may include a social media request to provide a reward 840 that may be interacted with by any of a plurality of second users in the first user's social media network. The post 840 may include alphanumeric characters, audio video content, and a variety of other things that indicate the first user has satisfied performance indicator. In the embodiment illustrated in FIG. 6W, the post includes a statement indicating the first user has satisfied a goal. The post may include alphanumeric characters, audio/visual content, and other information. The social media response to the request to provide a reward or post may be provided by the icon or emoji 860 or other interaction provided by the user. In the interface 600w, the response provided by the second user is an icon 860 which indicates the second user's affirmative approval or positive response of what the second one response communication to the request to provide the reward.

The response communication from the social media network may be provided for each of the plurality of users that interacted with post. The response communication may include the second user identifier identifying what second user interacted with the message and it may also include the content, icon, message, audio/visual content, or other information etc., that the second user input to the social media feed and post 851. The response communication may be sent as a data packet 231 from the social media network to the server (as in step 391 illustrated in FIG. 3D). The content may include both an approval and a denial communication. The server may include text recognition software, icon recognition software or other software that is configured to categorize whether the content is either an approval communication or denial communication. After determining if the second user content from the social media post is to be an approval communication or a denial communication, the information may be stored in the first user record. Additionally, the software may use this information an aggregate this information in order to determine if a threshold amount of second user want to provide a reward to the first user (akin to step 343). In certain embodiments, the system may send a request for payment to each of these second users that provided a positive reaction or interaction with the post on social media. In other embodiments, a payment request may be sent to the third user. Other embodiments for employing social media networks may be used and are within the spirit and scope of the present invention.

e. Artificial Intelligence and Neural Networks

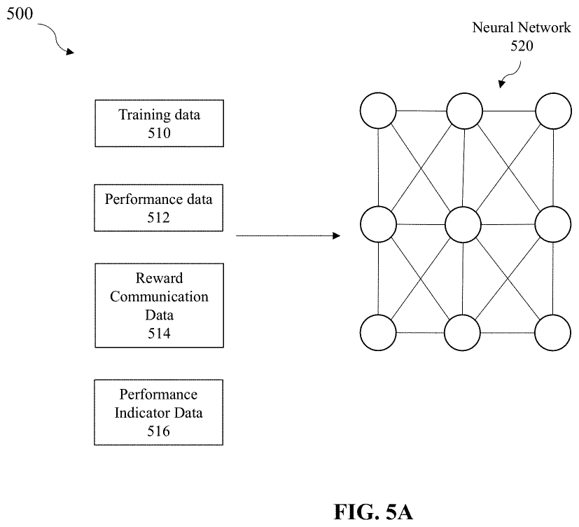

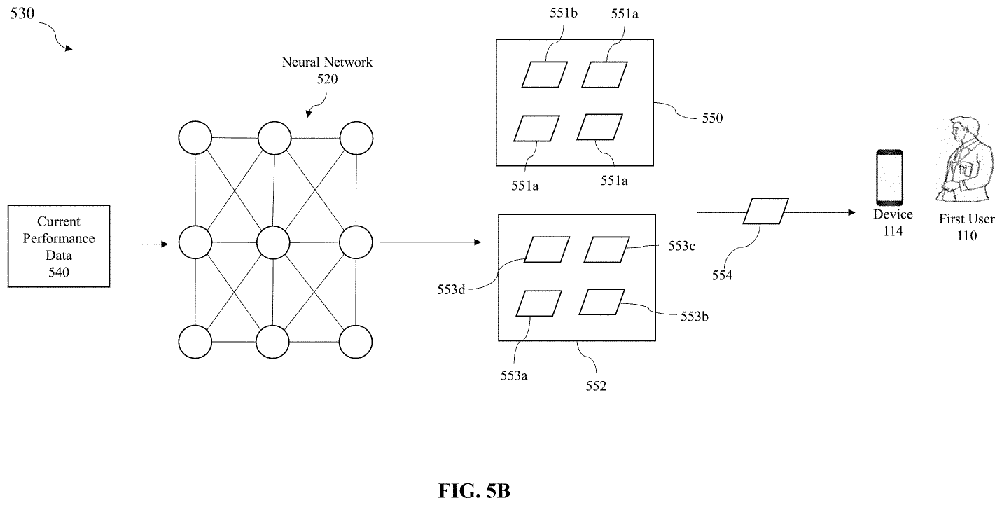

Artificial intelligence (e.g., machine learning) may be used with the systems and methods described herein. For instance, machine learning algorithms may be used to predictively produce communications that are most likely to cause a first user to produce a measured change. For instance, and referring now to FIG. 5A, a method 500 for training a neural network 520 is shown. As illustrated, the diagram shows processing training data 510 and performance data 512, reward communication data 514 and performance indicator data to generate neural network 520. It is understood that the reward communication data may include all the data received related to the unexpected reward communications and loss aversion communications sent to the first user . It is understood that the performance indicator data 516 may include all the performance indicator data and goal data that was stored in the first user record in the attached database.

The neural network may be stored in an attached database. Furthermore, the neural network may predict at least one of frequency, timing, content, and magnitude of the communication (e.g., unexpected reward, loss aversion) that is most likely to cause the first user to provide performance data that satisfies the performance indicator.

Referring now to FIG. 5B, a method 530 for using the neural network 520 to produce predictive communications that are most likely to cause the first user to provide performance data that satisfies the performance indicator is shown. As illustrated, current performance data 540 is used to calculate, using the neural network 520, a plurality of predicted communications (550, 552). The predicted communications can be one of an unexpected reward communication 550 or a loss aversion communication 552. As illustrated, the plurality of predictive unexpected reward communications 550 includes a first, second, third, and fourth unexpected reward communication (551a-55d). The plurality of predicted loss aversion communications similarly includes four predicted loss aversion communications (553a-553d). Regardless, a selected communication 554 is sent to the first user's 110 device 114.

The selected communication 554 may be the communication that is calculated (e.g., a probability of satisfying the performance indicator) to be the most likely to cause the first user to provide performance data that satisfies the performance indicator. Alternatively, the selected communication may be selected using a value-weighted shuffle (e.g., where the value is the probability of satisfying the performance indicator).

Thus, in one embodiment, a method comprises calculating, using the neural network, a plurality of predictive communications that are most likely to cause the first user to provide performance data that satisfies the performance indicator, selecting a predictive communication from the plurality of predictive communications, and sending, over the communication network, the predictive communication to the first user computing device.

Types of neural networks that may be generated from the training messages may include perceptron, feed forward, radial basis network, deep feed forward, recurrent neural network, long/short term memory, gated recurrent unit, auto encoder, variational auto encoder, denoising autoencoder, sparse autoencoder, Markov chain, Hopfield network, Boltzmann machine, restricted Boltzmann machine, deep belief network, deep convolutional network, deconvolutional network, deep convolutional inverse graphics network, deep residual network, Kohonen Network, Support Vector Machine, and Neural Turing Machine, among others. However, other types of neural networks may be used and are within the spirit and scope of the present invention.

iii. Devices

As referenced above, the methods described may be implemented on at least one processor. Moreover, a plurality of devices (e.g., first user device(s), second user device(s), third user device(s), etc.) may be involved in the methods and systems. The at least one processor may be included as a part of a computing device or may also be a device performing some or all of functions of a computing device. Referring now to FIG. 7, a computing device 700 is shown. FIG. 7 is a block diagram of a system including an example computing device 700 and other computing devices. Consistent with the embodiments described herein, the aforementioned actions performed by the servers and devices may be implemented in a computing device, such as the computing device 700 of FIG. 7. Any suitable combination of hardware, software, or firmware may be used to implement the computing device 700. Furthermore, computing device 700 may comprise or be included in the operating environment (e.g., shown by system 100) and processes and dataflow as described above. However, processes described above may operate in other environments and are not limited to computing device 700. Furthermore, computing device 700 may comprise an operating environment for system 100. Processes, data related to system 100 may operate in other environments and are not limited to computing device 700.

A system consistent with the invention may include a plurality of computing devices, such as computing device 700. In a basic configuration, computing device 700 may include at least one processing unit 702 and a system memory 704. Depending on the configuration and type of computing device, system memory 704 may comprise, but is not limited to, volatile (e.g., random access memory (RAM)), non-volatile (e.g., read-only memory (ROM)), flash memory, or any combination or memory. System memory 704 may include operating system 705, and one or more programming modules 706. Operating system 705, for example, may be suitable for controlling computing device 700′s operation. In one embodiment, programming modules 706 may include, for example, a program module 707 for executing the actions of system 100. Furthermore, embodiments of the invention may be practiced in conjunction with a graphics library, other operating systems, or any other application program and is not limited to any particular application or system. This basic configuration is illustrated in FIG. 7 by those components within a dashed line 720.

Computing device 700 may have additional features or functionality. For example, computing device 700 may also include additional data storage devices (removable and/or non-removable) such as, for example, magnetic disks, optical disks, or tape. Such additional storage is illustrated in FIG. 7 by a removable storage 709 and a non-removable storage 710. Computer storage media may include volatile and nonvolatile, removable, and non-removable media implemented in any method or technology for storage of information, such as computer readable instructions, data structures, program modules, or other data. System memory 704, removable storage 709, and non-removable storage 710 are all computer storage media examples (i.e., memory storage.) Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information, and which can be accessed by computing device 700. Any such computer storage media may be part of system 700. Computing device 700 may also have input device(s) 712 such as a keyboard, a mouse, a pen, a sound input device, a camera, a touch input device, etc. Output device(s) 714 such as a display, speakers, a printer, etc. may also be included. The aforementioned devices are only examples, and other devices may be added or substituted.

Computing device 700 may also contain a communication connection 716 that may allow system 100 to communicate with other computing devices 718, such as over a network 170 in a distributed computing environment, for example, an intranet or the Internet. Communication connection 716 is one example of communication media. Communication media may typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer readable media as used herein may include both computer storage media and communication media.

As stated above, a number of program modules and data files may be stored in system memory 704, including operating system 705. While executing on processing unit 702, programming modules 706 (e.g., program module 707) may perform processes including, for example, one or more of the stages of a process. The aforementioned processes are examples, and processing unit 702 may perform other processes. The aforementioned processes are examples, and processing unit 702 may perform other processes and may also be configured to provide graphical user interfaces displayed associated with devices explained above. Other programming modules that may be used in accordance with embodiments of the present invention may include electronic mail and contacts applications, activity tracking applications, word processing applications, spreadsheet applications, database applications, slide presentation applications, drawing or computer-aided application programs, etc.

Generally, consistent with embodiments of the invention, program modules may include routines, programs, components, data structures, and other types of structures that may perform particular tasks or that may implement particular abstract data types. Moreover, embodiments of the invention may be practiced with other computer system configurations, including hand-held devices, multiprocessor systems, microprocessor-based or programmable consumer electronics, minicomputers, mainframe computers, and the like. Embodiments of the invention may also be practiced in distributed computing environments where tasks are performed by remote processing devices that are linked through a communications network. In a distributed computing environment, program modules may be located in both local and remote memory storage devices.

Furthermore, embodiments of the invention may be practiced in an electrical circuit comprising discrete electronic elements, packaged, or integrated electronic chips containing logic gates, a circuit utilizing a microprocessor, or on a single chip (such as a System on Chip) containing electronic elements or microprocessors. Embodiments of the invention may also be practiced using other technologies capable of performing logical operations such as, for example, AND, OR, and NOT, including but not limited to mechanical, optical, fluidic, and quantum technologies. In addition, embodiments of the invention may be practiced within a general-purpose computer or in any other circuits or systems.

Embodiments of the present invention, for example, are described above with reference to block diagrams and/or operational illustrations of methods, systems, and computer program products according to embodiments of the invention. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments of the invention have been described, other embodiments may exist. Furthermore, although embodiments of the present invention have been described as being associated with data stored in memory and other storage mediums, data can also be stored on or read from other types of computer-readable media, such as secondary storage devices, like hard disks, floppy disks, or a CD-ROM, or other forms of RAM or ROM. Further, the disclosed methods' stages may be modified in any manner, including by reordering stages, and/or inserting or deleting stages, without departing from the invention.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

We claim:

1. A system for providing a first user an unexpected reward communication for a measured change, the system comprising one or more processors communicably connected to a communication network, the one or more processors including at least one processor configured for:
a. receiving, over the communications network, from an information source computing device, training data comprising metrics associated with an attribute of the first user;
b. creating a first user record for the first user;
c. storing the training data in the first user record;
d. determining a performance indicator for the metrics associated with the attribute of the first user;
e. receiving, over the communications network, from the information source computing device, performance data comprising the metrics associated with the attribute of the first user;
f. receiving, over the communications network, from a validation computing device, validation data for validating at least a portion of the performance data associated with the attribute;
g. determining if at least a portion of the performance data was validated using the validation data;
wherein if the portion of the performance data was validated using the validation data, the portion of the performance data is validated performance data;
h. determining if the validated performance data satisfies the performance indicator;
i. sending, over the communications network, to a first user computing device a communication; and,
wherein, if the performance data satisfies the performance indicator, the communication comprises an unexpected reward communication;
wherein, if the performance data does not satisfy the performance indicator, the communication comprises a loss aversion communication;
j. repeating step e-i until the validated performance data satisfies the performance indicator.

2. The system of claim 1, wherein at least one of the one or more processors is further configured for:
sending, over the communications network, to a second user computing device of one or more second users, a request to provide a reward to the first user if the validated performance data satisfies the performance indicator;
receiving, over the communications network, from the at least one second user computing device, at least one response communication to the request to provide the reward;
wherein the at least one response communication comprises a second user identifier and an approval communication;
selecting, from the at least one response communications, a provider of the unexpected award communication, wherein the provider is one of the second users;
sending, over the communications network, to the first user computing device, the unexpected reward communication, where the unexpected reward communication includes a second user identifier of the provider of the unexpected award communication.

3. The system of claim 2, wherein at least one of the one or more processors is further configured for, prior to the sending the second user computing device the request to provide the reward:
receiving, over the communications network, from one of the first user computing device, the second user computing device, and a third user computing device, second user contact data associated with each of the second users;
wherein the second user contact data comprises a second user contact name and at least one of a second user email address and a second user mobile phone number; and,
storing, in a second user record in an attached database, the second user contact data.

4. The system of claim 3, wherein before the receiving the validation data, the method comprises:
providing, over the communications network, to at least one of the first user computing device, the second user computing device and the third user computing device, a first interface for receiving validation data.

5. The system of claim 1, wherein the training data comprises metrics associated with one of a health measurement, a labor measurement, an athletic measurement, a scholastic measurement, a biological measurement, and an identifying status.

6. The system of claim 2, wherein the information source computing device comprises at least one of the first user computing device, the second user computing device, the third user computing device, a wearable device processor, and a sensor processor associated with a sensor.

7. The system of claim 1, wherein at least one of the one or more processors is further configured for:
processing the training data and performance data to generate a neural network; and
storing, in the attached database, the neural network;
wherein the neural network predicts at least one of frequency, timing, content, and magnitude of (1) loss aversion communications and (2) unexpected reward communications that are most likely to cause the first user to provide performance data that satisfies the performance indicator.

8. The system of claim 6, wherein at least one of the one or more processors is further configured for:
calculating, using the neural network, a plurality of predictive communications that are most likely to cause the first user to provide performance data that satisfies the performance indicator;
wherein the plurality of predictive communications comprise at least one of one or more unexpected reward communications and one or more loss aversion communications;
selecting a predictive communication from the plurality of predictive communications; and
sending, over the communication network, the predictive communication to the first user computing device.

9. The system of claim 1, wherein the measured change comprises at least one of a sleep pattern, a fat percentage, a muscle mass change, a change in reading habits, adherence to exercise regimens, adherence to therapy regimens, adherence to medication regimens, a respiratory change, or any other sustained change in behavior or biological variable captured by the system.

10. The system of claim 3, wherein the determining the performance indicator comprises applying a first function to the training data to determine a goal to be attained by the first user.

11. The system of claim 10, wherein at least one of the one or more processers is further configured for:
prior to sending the unexpected reward communication, sending, to the one or more second users, the request to provide the reward to the first user;
wherein the sending the request to provide the reward to the first user is in accordance with a request frequency;
wherein the request frequency is proportionally related to a difference between the performance data and the goal, wherein a frequency constant for calculating the request frequency is provided by the third user computing device.

12. The system of claim 10, wherein the unexpected reward communication comprises a reward magnitude that is proportionally related to a difference between the performance data and the goal, wherein a magnitude constant for calculating the reward magnitude is provided by at least one of the second user computing device and the third user computing device.

13. The system of claim 10, wherein the unexpected reward communication is provided to the first user according to a reward frequency that is proportionally related to a difference between the performance data received and the goal, wherein a frequency constant for calculating the reward frequency is provided by at least one of the second user computing device and the third user computing device.

14. The system of claim 10, wherein determining the performance indicator comprises:
sending, over the communications network, to at least one of the second user computing device and the third user computing device, a goal request for goal data for the goal of the first user;
receiving, over the communications network, from at least one of the second user computing devices and the third user computing device, the goal data for the goal of the first user; and
storing, in an attached database, the goal data for the first user;
wherein the goal data comprises an improvement over the training data.

15. A system for providing to a first user an unexpected reward communication for a measured change, the system comprising one or more processors communicable connected to a communication network, the one or more processor including at least one processor configured for:
a. receiving, over the communications network, from an information source computing device, training data comprising metrics associated with an attribute of the first user;
b. creating a first user record for the first user;
c. storing the training data in the first user record;
d. determining a performance indicator for the metrics associated with the attribute of the first user;
e. receiving, over the communications network, from the information source computing device, performance data comprising the metrics associated with the attribute of the first user;
f. sending, over the communications network, to the first user computing device, a communication;
wherein, if the performance data satisfies the performance indicator, the communication comprises an unexpected reward communication;
wherein, if the performance data does not satisfy the performance indicator, the communication comprises a loss aversion communication;
g. repeating step e and f until the validated performance data satisfies the performance indicator.

16. The system of claim 15, wherein at least one of the one or more processors is further configured for:
sending, over the communications network, to a second user computing device of one or more second users, a request to provide a reward to the first user if the performance data satisfies the performance indicator;
receiving, over the communications network, from the at least one second user computing device, at least one response communication to the request to provide the reward;
wherein the at least one response communication comprises a second user identifier and an approval communication;
selecting, from the at least one response communications, a provider of the unexpected reward communication, wherein the provider is one of the second users;
sending, over the communications network, to the first user computing device, the unexpected reward communication, where the unexpected reward communication includes a second user identifier of the unexpected reward communication.

17. The system of claim 16, wherein determining the performance indicator comprises:
sending, over the communications network, to at least one of the second user computing devices and a third user computing device, a goal request for goal data for the goal of the first user;
receiving, over the communications network, from at least one of the second user computing devices and the third user computing device, the goal data for the goal of the first user, where the goal data comprises an improvement over the training data; and, storing, in the attached database, the goal data for the first user.

18. The system of claim 16, wherein at least one of the one or more processors is further configured for, prior to the sending the second user computing device the request to provide the reward:
receiving, over the communications network, from one of the first user computing device, the second user computing device and a third user computing device, second user contact data associated with each of the second users;
wherein the second user contact data comprises a second user contact name and at least one of a second user email address and a second user mobile phone number; and,
storing, in a second user record in an attached database, the second user contact data.

19. A computer implemented method executed by one or more computing devices for providing to a first user an unexpected reward communication for a measured change, the method comprising:
a. receiving, from an information source computing device, training data comprising metrics associated with an attribute of the first user;
b. creating a first user record for the first user;
c. storing the training data in the first user record;
d. determining a performance indicator for the metrics associated with the attribute of the first user;
e. receiving, from the information source computing device, performance data comprising the metrics associated with the attribute of the first user;
f. sending, to the first user computing device, a communication;
wherein, if the performance data satisfies the performance indicator, the communication comprises an unexpected reward communication;
wherein, if the performance data does not satisfy the performance indicator, the communication comprises a loss aversion communication; and,
g. repeating step e and f until the validated performance data satisfies the performance indicator.
